(19) United States
(12) Patent Application Publication
(10) Pub. No.: US 2022/0411886 A1
(43) Pub. Date: Dec. 29, 2022

(54) Methods and Systems for Detection of Covid Variants

(71) Applicant: Laboratory Corporation of America Holdings, Burlington, NC (US)

(72) Inventors: Jonathan David Williams, Hillsborough, NC (US); Brian Krueger, Burlington, NC (US); Brian Matthew Norvell, Liberty, NC (US); Scott Parker, Cary, NC (US); Lakshmanan K. Iyer, North Attleboro, MA (US); Hayden N. Brochu, Charlotte, NC (US)

(21) Appl. No.: 17/845,629

(22) Filed: Jun. 21, 2022

Related U.S. Application Data
(60) Provisional application No. 63/213,110, filed on Jun. 21, 2021.

(51) Int. Cl. C12Q 1/70

(52) U.S. Cl. CPC C12Q 1/701; C12Q 1/6869

(57) ABSTRACT

Disclosed are methods and systems for the detection of variants of the SARS-CoV-2 virus that cause COVID-19. For example, disclosed are methods for identifying and/or tracking variants of SARS-CoV-2 comprising: (a) identifying a sample from a subject as positive for SARS-CoV-2 nucleic acid and/or antibodies to SARS-CoV-2; (b) generating a sample-specific SARS-CoV-2 nucleic acid from the sample; (c) performing nucleic acid sequencing on the sample-specific SARS-CoV-2 nucleic acid; and(d) determining whether the nucleic acid sequence comprises a SARS-CoV-2 variant sequence. Also disclosed are systems for performing any portion of the disclosed methods and computer-program products tangibly embodied in a non-transitory machine-readable storage medium, including instructions configured to perform any of the steps of the disclosed methods or run any portion of the disclosed systems.

### RELATED APPLICATIONS

This application claims priority to U.S. Provisional Patent Application No. 63/213,110, filed Jun. 21, 2021. The disclosure of U.S. Provisional Patent Application No. 63/213,110 is incorporated by reference in its entirety herein.

### FIELD

Disclosed are methods and systems for the detection of variants of the SARS-CoV-2 virus that cause COVID-19 and the geographic location of individuals infected with any strain of the variants.

### INTRODUCTION

SARS-CoV-2 is an enveloped, single-stranded RNA virus of the family Coronaviridae, genus Beta coronavirus. All coronaviruses share similarities in the organization and expression of their genome, which encodes 16 nonstructural. proteins and the 4 structural proteins: spike (S), envelope (E), membrane (M), and nucleocapsid (N). Viruses of this family are of zoonotic origin. They cause disease with symptoms ranging from those of a mild common cold to more severe ones such as the Severe Acute Respiratory Syndrome (SARS), Middle East Respiratory Syndrome (MFRS) and Coronavirus Disease 2019 (COVID-19). Other coronaviruses known to infect people include 229E, NL63, OC43 and HKU1, The latter are ubiquitous and infection typically causes common cold or flu-like symptoms.

The 2019 Novel Coronavirus (SARS-CoV-2) is a beta-coronavirus that first emerged as a pathogen with outbreak potential in Wuhan, China in December 2019. Initial reports suggested that limited person to person transmission occurred within China. However, in early 2020, additional cases of 2019-nCoV have been detected worldwide, indicating sustained person to person transmission. To date, the clinical spectrum of SARS-CoV-2 has ranged from mild, self-limiting upper respiratory tract infections to more serious lower respiratory tract illness leading to significant morbidity and mortality. As the SARS-CoV-2 pandemic has accelerated, more keen attention has been paid to diversity of viral genomic sequences, and how these variants may affect transmissibility of infection, severity of infection, or viral escape from natural or vaccine-induced immunity.

Viruses constantly change through mutation. Multiple variants of the virus that causes COVID-19 have been documented in the U.S. and globally. Some variants may emerge and disappear; other variants may persist and display increased infectivity or severity of symptoms. For example, as of June 2021 there were six notable variants in the United States. (1) B.1.1.7: this variant was first detected in the United States in December 2020. It was initially detected in the United Kingdom. (2) B.1.351: this variant was first detected in the United States at the end of January 2021 and was initially detected in South Africa in December 2020. (3) P.1: this variant was first detected in the United States in January 2021—P.1 was initially identified in travelers from Brazil, who were tested during routine screening at an airport in Japan, in early January. (4) B.1.427 and (5) B.1.429: these two variants were first identified in California in February 2021. (6) B.1.617.2: this variant was first detected in the United States in March 2021. It was initially identified in India in December 2020. CDC.gov/coronavirus/2019-ncov/variants.

Thus, there is a need to identify and track new variants. There is further a need to track the geographic location of infected individuals to assist public health authorities in responding to the pandemic.

### SUMMARY

Disclosed are methods and systems for identifying and tracking variants of SARS-CoV-2 that can cause COVID-19. The methods and systems may be embodied in a variety of ways.

In certain embodiments, the method may comprise a method for identifying and/or tracking variants of SARS-CoV-2 comprising the steps of: (a) identifying a sample from a subject as positive for SARS-CoV-2 nucleic acid and/or antibodies to SARS-CoV-2; (b) generating a sample-specific SARS-CoV-2 nucleic acid from the sample; (c) performing nucleic acid sequencing on the sample-specific SARS-CoV-2 nucleic acid; and (d) determining whether the nucleic acid sequence comprises a SARS-CoV-2 variant sequence.

In an embodiment, sequencing covers the majority of the viral genome. Thus, in certain embodiments, where the sample SARS-CoV-2 genome is amplified by RT-PCR, the resulting cDNA is then further amplified using tiled primers that bind at spaced intervals along the viral genome. In certain embodiments, the tiled primers are spaced such that adjacent primers are 600 bp apart from each other. In this way, the SARS-CoV-2 genome is amplified in a highly efficient manner regardless of the presence or absence of new variants. For example, in certain embodiments, the nucleic acid sequencing comprises sequencing at least 80%, or optionally at least 85%, or optionally at least 90%, or optionally at least 95% of the entire viral genome.

The amplified nucleic acid molecules may be labeled with molecular barcode identifying sequences. For example, in certain embodiments, the tiled primers are primers further comprise an adaptor for the addition of a barcode sequence and/or universal primer sites for nucleic acid sequencing.

Also disclosed are systems for performing any of the steps of the disclosed method steps as well as a computer-program product tangibly embodied in a non-transitory machine-readable storage medium, including instructions configured to run any of the stations and/or components of the system and/or perform a step or steps of the methods of any of the disclosed embodiments.

Also disclosed are systems that include one or more data processors and a non-transitory computer readable storage medium containing instructions which, when executed on the one or more data processors, cause the one or more data processors to perform part or all of one or more methods disclosed herein, and computer program products tangibly embodied in a non-transitory machine-readable storage medium, and that include instructions configured to cause one or more data processors to perform part or all of one or more methods disclosed herein.

The sequencing described herein is advantageous for identifying variants. A variety of nucleic acid sequencing protocols may be used. In certain embodiments, the nucleic acid sequencing comprises RT-PCR. For example, in certain embodiments, a PacBio® sequencing protocol and or apparatus is used.

In further embodiments, disclosed are methods and systems for identifying the geographic location of individuals infected with a variant. For example, in certain embodiments, the barcode is linked to the individual's zip code or other geographic identifier. In addition, the disclosure provides methods and/or systems to track the prevalence of variants in a population of infected individuals and/or a general population. In either case, a geographic region may comprise the population. In a further embodiment, the disclosure provides methods and systems to correlate specific variants with infectivity (virus transmission) and disease severity.

Data generated by a method or system of the disclosure may be combined with other data of a similar type from other sources and/or other data of a different type in analysis. In certain embodiments, data may be deposited in a depository for analysis and/or combination with other data. In certain embodiments, the depository is a CDC database. Or, other government or university or private databases may be engaged.

### FIGURES

The disclosure may be better understood by reference to the following non-limiting figures.

FIG. 1 shows a method for detection of SARS-CoV-2 variants in accordance with an embodiment of certain steps of the disclosure.

FIG. 2 shows a method for preparing a sample-specific SARS-CoV-2 nucleic acid for sequencing in accordance with an embodiment of certain steps of the disclosure.

FIG. 3 shows a method for whole genome sequencing and variant identification in accordance with an embodiment of the disclosure.

FIG. 4 shows method steps for analysis of sequence data in accordance with an embodiment of the disclosure.

FIG. 5 shows method steps for variant identification and lineage assignment in accordance with an embodiment of the disclosure.

FIG. 6 shows method steps for revalidation of variant identification using in-house data and an external database in accordance with an embodiment of the disclosure.

FIG. 7 shows a system for detection of SARS-CoV-2 variants in accordance with an embodiment of the disclosure.

FIG. 8 shows a computing device for use with any of the methods or systems in accordance with an embodiment of the disclosure.

FIG. 9 shows a map of a 96 well plate used to distribute M13 forward (1001-1032) and M13 reverse primers (1049-1079, and 1082) into amplification reactions in accordance with an embodiment of the disclosure.

FIG. 10 shows a map of a 96 well plate with combinations of M13 forward (1001-1032) and reverse (1049-1051) barcoded primers for use in amplification reactions in accordance with an embodiment of the disclosure.

FIG. 11 shows a distribution of average read coverage (NTC average CCS read depth) in accordance with an embodiment of the disclosure.

FIG. 12 shows confirmed negative mucleic acid amplification (NAA) diagnostic samples' average CCS read count in accordance with an embodiment of the disclosure.

FIG. 13 shows a distribution of strains in accordance with an embodiment of the disclosure.

FIG. 14 shows the rate of 90% genome coverage by NAA CT value in accordance with an embodiment of the disclosure.

FIG. 15 shows the average read count for inter-assay samples used in a stability study for three separate sequencing runs (PBT5073, PBT5075, and PBT5080) in accordance with an embodiment of the disclosure.

### DETAILED DESCRIPTION

Definitions

The terms sample or patient sample or biological sample or specimen are used interchangeably herein. Samples may include upper and lower respiratory specimens. Such specimens (samples) may include nasopharyngeal or oropharyngeal swabs, sputum, lower respiratory tract aspirates, bronchoalveolar lavage, and nasopharyngeal washes/aspirates or nasal aspirates. Other non-limiting examples of samples include, a tissue sample (e.g., biopsies), blood or a blood product (e.g., serum, plasma, or the like), cell-free DNA, urine, a liquid biopsy sample, or combinations thereof. The term “blood” encompasses whole blood, blood product or any fraction of blood, such as serum, plasma, buffy coat, or the like as conventionally defined.

As used herein, the term subject or individual refers to a human or any non-human animal. A subject or individual can be a patient, which refers to a human presenting to a medical provider for diagnosis or treatment of a disease, and in some cases, wherein the disease may be any infection by a pathogen. Also, as used herein, the terms “individual,” “subject” or “patient” includes all warm-blooded animals.

As used herein SMRT refers to single-molecule real-time sequencing that uses a zero-mode waveguide (ZMW). A single DNA polymerase enzyme is affixed at the bottom of a ZMW with a single molecule of DNA as a template. The ZMW creates an illuminated observation volume that is small enough to observe only a single nucleotide being incorporated. Each of the four DNA bases is attached to one of four different fluorescent dyes. When a nucleotide is incorporated by the DNA polymerase, the fluorescent tag is cleaved off and diffuses out of the observation area of the ZMW where its fluorescence is no longer observable. A detector detects the fluorescent signal of the nucleotide incorporation, and the base call is made according to the corresponding fluorescence of the dye.

As used herein, CT or ct refers to cycle threshold, or the total number of cycles required to amplify and detect a viral (e.g., SARS-CoV-3) nucleic acid by RT-PCR.

As used herein loci loop capture is the process of using molecular inversion probes to bind to and amplify a region of interest within the viral genome.

As used herein, CCS or circular consensus sequencing reads are processed reads that have been corrected for errors in raw sequencing data by sequencing the length of a captured DNA fragment multiple times.

As used herein, repeatability (or intra-assay precision) describes the closeness of agreement between results of successive measurements of the same analyte and carried out under the same conditions of measurement. Intra-assay repeatability is the measurement of the variability when the same specimen is analyzed during one analytical run.

As used herein reproducibility (or inter-assay precision) describes the closeness of agreement between results of successive measurements of the same analyte and carried out under the same conditions of measurement. Inter-assay repeatability is a measurement of the variability when the same specimen is analyzed during more than one run.

As used herein, concordance measures the closeness of agreement between the measured value and the value that is accepted as a conventional true or accepted reference value. This can require a “gold standard” or an accepted method to which a new method can be compared.

As used herein, analytical validity requires establishing the probability that a test will be positive when a particular sequence (analyte) is present (analytical sensitivity) and the probability that the test will be negative when the sequence is absent (analytical specificity). In next generation sequencing (NGS), analytical sensitivity can be the likelihood that the assay will detect the targeted sequence variations, if present nucleic acid sequences derived from the assay and a reference sequence. For NGS, analytical specificity is defined as the probability that the assay will not detect a sequence variation when none are present (the false detection rate is a useful measurement for sequencing assays).

As used herein, specificity defines the ability of a measurement procedure to measure solely the analyte.

As used herein, the assay tolerance for nucleic acid input is the tolerance to variation in the amount of analyte added to the reactions.

As used herein, GISAID is a global science initiative and primary source established in 2008 that provides open access to genomic data of influenza and coronavirus (e.g., COVID-19) data. The database has become the world's largest repository for SARS-CoV-2 sequences. GISAID facilitates genomic epidemiology and real-time surveillance to monitor the emergence of new COVID-19 viral strains.

As used herein, when an action is “based on” something, this means the action is based at least in part on at least a part of the something.

### Methods for NGS SARS-CoV-2 Strain Determination

Disclosed are methods and systems for identifying and tracking variants of SARS-CoV-2 that can cause COVID-19. The methods and systems may be embodied in a variety of ways.

In certain embodiments, the method may comprise a method for identifying and/or tracking variants of SARS-CoV-2 comprising the steps of: (a) identifying a sample from a subject as positive for SARS-CoV-2 nucleic acid and/or antibodies to SARS-CoV-2; (b) generating a sample-specific SARS-CoV-2 nucleic acid from the sample; (c) performing nucleic acid sequencing on the sample-specific SARS-CoV-2 nucleic acid; and (d) determining whether the nucleic acid sequence comprises a SARS-CoV-2 variant sequence.

The method may utilize samples for which the COVID status is not known, or may use samples that have previously tested positive for COVID. In certain embodiments, the positive samples may be identified using an approved EUA approved COVID-19 RT-PCR Test (e.g., Labcorp EUA200011 and/or EUA203057). In this way, results are for the identification of the SARS-CoV-2 strain infecting an individual after detection of viral RNA in the sample.

In an embodiment, sequencing covers the majority of the viral genome. Thus, in certain embodiments, where the sample SARS-CoV-2 genome is amplified by RT-PCR, the resulting cDNA is then further amplified using tiled primers that bind at spaced intervals along the viral genome. In certain embodiments, the tiled primers are spaced such that adjacent primers are 600 bp apart from each other. In this way, the SARS-CoV-2 genome is amplified in a highly efficient manner regardless of the presence or absence of new variants. For example, in certain embodiments, the nucleic acid sequencing comprises sequencing at least 80%, or optionally at least 85%, or optionally at least 90%, or optionally at least 95% of the entire viral genome.

The amplified nucleic acid molecules may be labeled with molecular barcode identifying sequences. For example, in certain embodiments, the tiled primers are primers further comprise an adaptor for the addition of a barcode sequence and/or universal primer sites for nucleic acid sequencing.

In certain embodiments, the step of generating a sample-specific SARS-CoV-2 nucleic acid comprises using reverse transcriptase polymerase chain reaction (RT-PCR) to generate a sample-specific SARS-CoV-2 cDNA.

Also, in certain embodiments, the step of generating a sample-specific SARS-CoV-2 nucleic acid comprises using a targeted next-generation sequencing in combination with inverted molecular probes as a way to generate the sample-specific SARS-CoV-2 nucleic acid (e.g., Molecular Loop SARS-CoV-2 Sequencing Panel). For example, in certain embodiments the step of generating a sample-specific SARS-CoV-2 nucleic acid further comprises hybridizing one strand of the sample SARS-CoV-2 cDNA to a single-stranded probe DNA template comprising a pair of SARS-CoV-2 probes, wherein the first probe is positioned at the 3′ end of the probe DNA template and the second probe is positioned at the 5′ end of the probe DNA template. In this way, the 3′ probe functions as a forward primer and the 5′ probe functions as a reverse primer.

In certain embodiments, the probe sequences are selected as tiled probes that bind at spaced intervals along a SARS-CoV-2 genome. In an embodiment, the Wuhan-Hu-1 SARS-CoV-2 reference genome (NC_045512) (available at www.ncbi.nlm.nih.gov/nuccore/NC_045512) is used. Or, other known reference genomes may be used. For example, in alternate embodiments, the probes may be spaced by about 100, or 200, or 300, or 400, or 500, or 600, or 700, or 800, or 900 or more than 1,000 base pairs. Or, spacings within this range (e.g., 450, 550, 650 or 750) may be used. The probes may be tiled across greater than 99% (e.g., 99.6%) of the 30 kb SARS-CoV-2 viral genome. The probes may be tiled over and/or to provide a sequence on average for a given nucleotide 2X, 7X, 22X or more.

Also, in certain embodiments, the single-stranded probe DNA template further comprises universal sequencing primers (e.g., M13 primers) positioned adjacent to the probe sequences. These can allow for enrichment with matching universal primer sequences and unique sample specific barcoding for downstream bioinformatic analysis. Additionally, in certain embodiments, and as disclosed in more detail herein, the single-stranded probe DNA template further comprises an adaptor sequence for the addition of a barcode sequence used to correlate the SARS-CoV-2 sample-specific nucleic acid to a sample number. In some cases, the barcode may be correlated to the zip code from which the sample and/or patient originated. Also, the method may include filling in the sequence between the two probes to generate a circular single-stranded probe DNA template comprising sequence specific to the sample SARS-CoV-2 cDNA between the two probe sequences and then releasing the circular single-stranded probe DNA template comprising sequence specific to the sample SARS-CoV-2 cDNA from the sample-specific SARS-CoV-2 DNA and digestion of the circular single-stranded probe DNA template comprising sequence specific to the sample SARS-CoV-2 cDNA to generate a linear DNA used as a template for nucleic acid sequencing. In certain embodiments, the linear probe DNA template is then modified to add adaptors and then PCR amplified (enriched) for DNA sequencing. In certain embodiments, the step of enrichment comprises a purification step (e.g., bead purification). For example, in certain embodiments, the substrate for sequencing is generated by RT-PCR and then SARS-CoV-2 sequences identified using ˜1000 tiled Molecular Loop Inversion Probes (MIPS) designed to amplify RNA that has been reverse transcribed to cDNA from 99.6% of the SARS-CoV-2 genome with most bases covered by 22 MIPs. In certain embodiments, the product synthesized in-between the MIPS is enriched and has sample specific molecular barcodes added via amplification followed by sequencing.

In certain embodiments, the method employs whole genome sequencing. In certain embodiments, next generation sequencing (NGS) is used. Or, other types of sequencing such as but not limited to Sanger sequencing, shot gun sequencing, SMRT sequencing, pyrosequencing or nanopore sequencing may be used. For example, in certain embodiments the PacBio whole genome sequencing with the corresponding SMRT link 9 software and analysis tools may be used. For example, in one embodiment, the method may employ a PacBio whole genome sequencing test for SARS-CoV-2 strain identification using residual total nucleic acid extracts from positive samples. In certain embodiments, the nucleic acid sequencing comprises sequencing at least 80%, or optionally 85%, or optionally 90% or greater of the entire viral genome.

In certain embodiments, the step of determining whether the nucleic acid sequence comprises a SARS-CoV-2 variant sequence comprises aligning the sample SAR-CoV-2 sequence to a SARS-CoV-2 reference genome to generate a sample-specific assembly and consensus sequence. Additionally, the method may comprise assessing the lineage for the sample. In certain embodiments, the method may include identifying the geographic location of the subject.

Additionally, as disclosed herein, in certain embodiments, the method may include uploading the results of the step of determining whether the nucleic acid sequence comprises a SARS-CoV-2 variant sequence into a depository for further classification (e.g., lineage determination) if a variant is detected. The depository may be a CDC database. Or, other public depositories may be used.

The method may further include determining if an update to the depository has been made prior to the step of determining whether the nucleic acid sequence comprises a SARS-CoV-2 variant sequence.

The method may be automated at various steps in the procedure. In certain embodiments, the method may be used with Hamilton Star robots for sample plate setup. Additionally, and/or alternatively, Formulatrix Mantis Liquid Handlers or other automated devices may be used for mastermix distribution. Also, as disclosed herein the method may be computer implemented and/or include use of a computer-program product tangibly embodied in a non-transitory machine-readable storage medium, including instructions configured to perform any of the steps of the method.

For example, in certain embodiments, residual total nucleic acid extract from SARS-CoV-2 positive RT-PCR diagnostic testing samples with Ct values <31 are cherry picked, e.g., as disclosed in more detail herein, from RNA extraction plates into a 96 well plate containing only positive samples using Hamilton STARs. Samples may then be aliquoted into a sequencing run plate of 95 samples with one water non-template control (NTC). The method may be scaled as required. For example, in certain embodiments, eight plates, or 760 specimens, may be processed in one production batch.

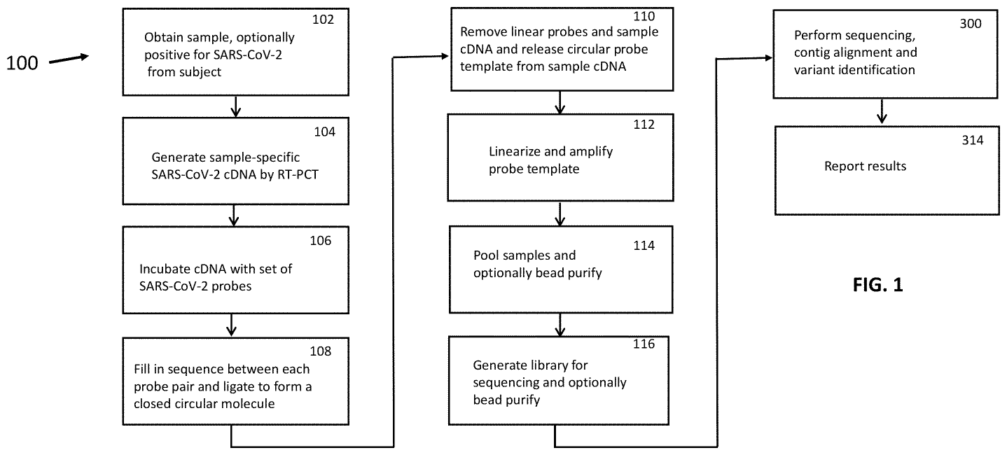

FIG. 1 shows an example of an embodiment of a method 100 of the disclosure. As illustrated in FIG. 1, a sample for testing, optionally positive for SARS-CoV-2, is obtained from a subject 102. In an embodiment, SARS-CoV-2 cDNA sequences are generated by RT-PCT 104. The SARS-CoV-2 cDNA may then be incubated with a set of tiled probes 106. In certain embodiments, the tiled probes are relatively evenly spaced across the SARS-CoV-2 genome. After binding, the region in-between the two probes may be filled in with DNA polymerase and ligated to form a closed circular molecule having sample-specific SARS-CoV-2 nucleic acid sequences positioned between the two probe sequences (i.e., a circular probe template) 108. Non-binding or incomplete loops remain linear molecules and may be removed with exonuclease digestion 110. Also at this point, the circular probe template molecules may be released from the sample cDNA (i.e., during denaturation in the PCR amplification reaction) 110. The probe may then be linearized, and enriched via amplification with a 3′ M13 universal sequence and 5′ sample specific barcodes 112. Samples are then pooled by equal volume 114 and library prepped for sequencing 116. At this point, samples are sequenced as disclosed herein and results analyzed 300 and reported 400.

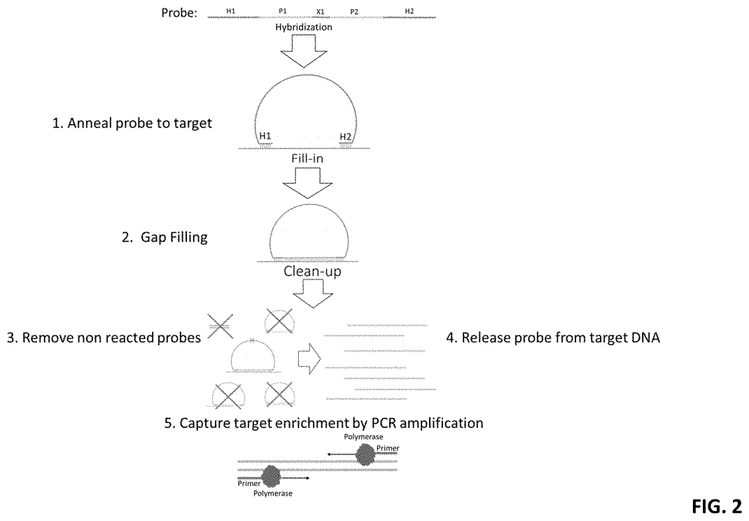

FIG. 2 provides an alternate illustration of the portion of the method for capturing SARS-CoV-2 specific sequences for sequence analysis. Thus, in certain embodiments, a custom Molecular Loop SARS-CoV-2 Capture Kit is used to prepare the samples to sequence. In certain embodiments, as disclosed herein, sequencing is performed using PacBio Sequel II sequencer. Thus, reverse transcriptase is used to synthesize cDNA from RNA. The SARS-CoV-2 cDNA is then used as a target for hybridization of molecular loop probes (FIG. 2, Step 1). Molecular loop probes may be tiled across greater than 99% (e.g., 99.6%) of the 30 kb SARS-CoV-2 viral genome and consist of two binding sites approximately 600 bp apart. After binding, the region in-between the two probes is synthesized with DNA polymerase and ligated to form a closed molecule (FIG. 2, Step 2). Non-binding or incomplete loops remain linear molecules and are removed with exonuclease digestion (FIG. 2, Step 3). Circular molecules are then released from the template cDNA (FIG. 2, Step 4) and then linearized (e.g., by digestion at X1) and enriched via amplification with a 3′ molecular loop specific M13 universal sequence and 5′ sample specific barcodes (FIG. 2, Step 5). P1 and P2 in FIG. 2 are adaptors that may be used for barcode addition. Samples are then pooled by equal volume and library prepped for PacBio sequencing. Library preparation may entail DNA damage repair, ligation of sequencing adapters, non-ligated product removal by enzymatic digestion, and bead purification. Libraries may then sequenced on a PacBio Sequel II, e.g., with 15 hour movies.

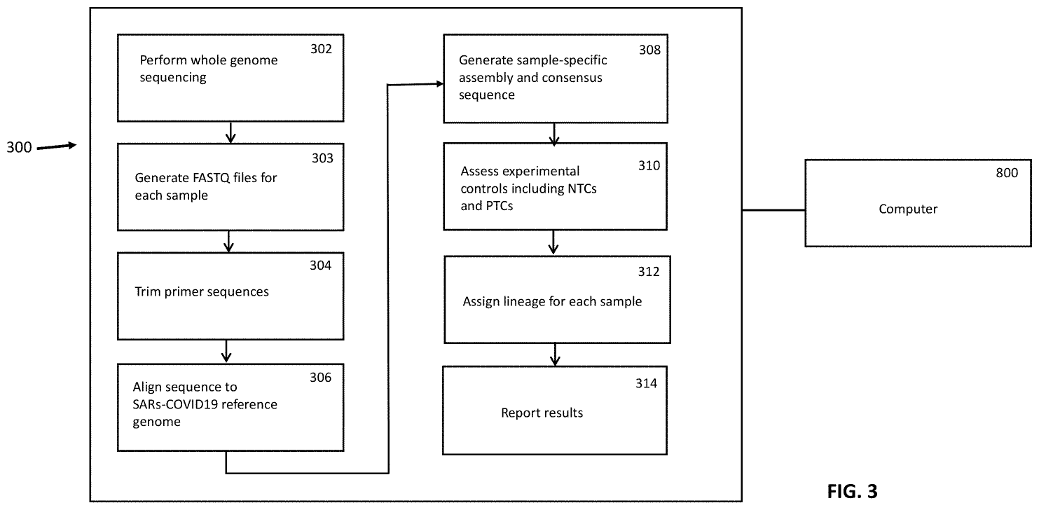

As illustrated in FIG. 3, at this point the steps of sequencing and data analysis may be performed 300. Thus, in certain embodiments, the method may comprise computer-implemented steps for sequence analysis. In certain embodiments, whole genome sequencing is performed 302 In certain embodiments, the sequencing utilizes Single Molecule, Real-Time (SMRT) long-read sequencing technology. The circular template generated from library prep is bound with a polymerase and primer and loaded onto the SMRTCell (sequencing cell). A single molecular product diffuses into one of 8 million zero-mode waveguide (ZMWs) wells where the polymerase is immobilized at the bottom. Phospholinked nucleotides are then introduced to the ZMWs where the base can then be incorporated by the polymerase. When a given base pair is incorporated, its addition produces a nucleotide specific emission of light that is detected on a per well basis by a camera. This process is repeated for a given amount of time, or movie length, and the nucleotide order on a given well is analyzed and translated to the corresponding nucleotide in the long sequence read output.

Next, the data may be assembled as sequence files 303. For example, in certain embodiments, PacBio SMRT LINK software and custom molecular loop processing scripts may be used to generate the FASTQ files for each sample. FASTQs may be analyzed using a genome analysis pipeline implemented using a CLC genomics server version 6.5.6. Or, other sequencing analysis systems may be used. At this point, the sequencing primer sequences can be removed 304 and the sequence aligned to a SARS-CoV-2 reference genome (e.g., NC_045512v2) to generate a bam file of alignment 306. In certain embodiments, Minimap2 may be used to generate the alignment. Or, other alignment programs may be used. In an embodiment, samples meeting minimum coverage of 50% are then used as the input for calling variants and for generating a sample-specific genome assembly to generate a consensus sequence for each sample 308. Or, other minimum coverage limits (e.g., 20, 30, 40, 60, 70, 80, 90 percent) may be used. In an embodiment, the consensus sequence may be generated using VCFcons (available at www.biorxiv.org/content/10.1101/2021.02.26.433111v1). Or, another algorithm may be used. In an embodiment, there is a defined threshold for generating the consensus sequence. For example, in certain embodiments, when VCFcons calls a nucleotide sequence for genome construction it must have at least 4 circular consensus sequencing (CCS) reads covering that base pair and an alternate allele frequency compared to the reference of >50%. If a nucleotide has less than 4 reads it is reported as N (a non-defined nucleotide) in the consensus sequence.

Assignment of sample lineage may take into account certain experimental variables and/or controls 310. For example, in certain embodiments, evaluation of an external no template control (NTC) is used to assess the validity of the results 310. Additionally and/or alternatively, an external positive template control (PTC) may be added to verify adequate processing of the plate 310. Further, in certain embodiments, unique strains (as available) from successful runs can be pooled by strain type and each unique pooled strain can be added to plates across a batch (e.g., a set of 8 sequencing plates) to ensure plate provenance across plate processing. An external non-template control (NTC) may be needed to ensure master mix contamination events are not present on the given amplification plate. The NTC may comprise water (e.g., molecular grade water) added to a defined position (e.g., the A1 position) of every 96 well positive plate before sample addition. Or, other NTCs (e.g., buffer) may be used. The NTC is may then be transferred along with positive samples to the sequencing run plate and taken through sequencing and (quality control) QC analysis.

In certain embodiments, after sequencing, the strain typing of a given plates positive control can be compared to the documented strain added before processing. Any discordance between a plates assigned strain typing can be further investigated to determine whether to proceed with the individual plate. For example, in certain embodiments, an inability to reconcile the positive control result can result in removal of all strains associated with a given control's plate. In other embodiments, a failed reaction of positive control will not necessarily lead to removal of results if the corresponding controls in other plates in the batch can rule out potential plate swaps.

In certain embodiments, after sequencing and NTC analysis the mean of medium CCS reads may be computationally analyzed for passing acceptance criteria of 10 CCS reads 310. In certain embodiments, for a positive sample's results to be released for a given 96 well sequencing plate the NTC must return a mean of median of a defined level (e.g., <10) CCS reads. If a plate's given NTC's mean of median CCS reads is greater than the defined level of CCS reads, all corresponding samples on the plate may be scheduled to be repeated.

At this point, lineages for individual samples may then be assigned using the consensus sequence 312. In an embodiment, this is performed as input to the Pangolin analysis package. Or, other analyses may be used. In certain embodiments, strain lineage results are released for samples with 90% genome coverage and/or whose mean of median read coverage across the whole genome is >10 circular consensus sequence (CCS) reads 314. In an embodiment, the different CCS read metrics are based on the nucleotide level (4 CCS reads) and on the genome level (10 CCS reads).

In certain embodiments, assessment of the strain determination results are performed after NTC analysis and removal of any samples on a plate with a failed NTC. Individual sample results are then computationally investigated for mean of median CCS reads >10 CCS and percent genome coverage is >90%. In certain embodiments, test results may be reported to healthcare providers and relevant public health authorities in accordance with local, state, and federal requirements. In certain embodiments, samples not meeting these criteria fail analysis and strain typing is not reported. Additionally and/or alternatively, when only positive samples are tested, the method is not used for detection of SARS-CoV-2 infection status where infection status is not dictated by viral whole genome sequencing results.

Data Analysis

The analysis of the sequence data may, in certain embodiments, comprise a pre-processing (i.e., upstream) steps and post-processing (i.e., downstream) steps. In certain embodiments, at least some of these steps comprise computer-implemented steps for data analysis. The upstream analysis may comprise monitoring the sequencer runs for completion, demultiplexing to generate individual sample FASTQ files, and triggering the alignment of each to the SARS-CoV-2 reference genome to generate alignments and variant call. The downstream analysis for samples in each SMRTCell may be comprised of generating all the results including the lineage classifications for each sample.

Upstream Analysis

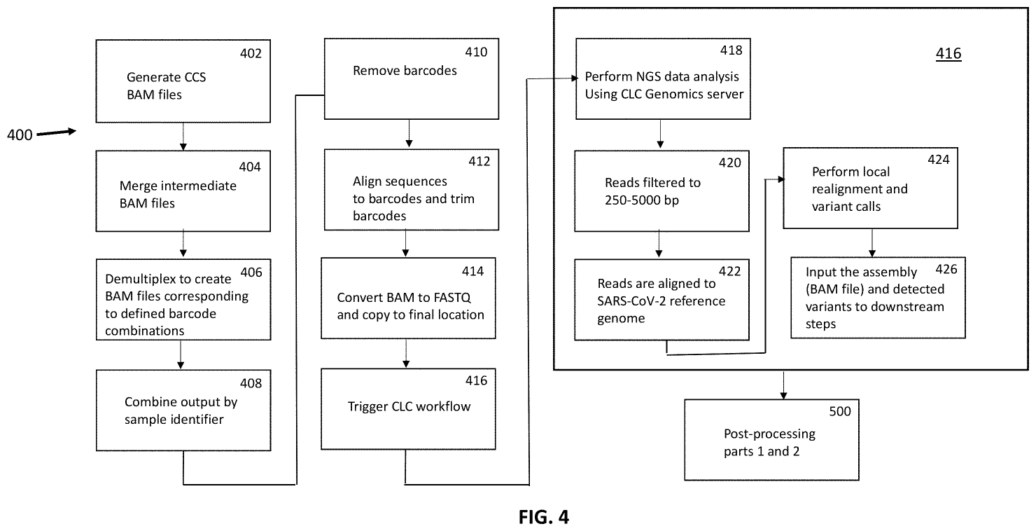

An example method for upstream analysis 400 of the sequencing data is shown in FIG. 4. Thus, in certain embodiments, for the analysis invocation, PacBio/Molecular Loop raw sequencing data may be deposited and a CCS BAM file created copied for demultiplexing. In an embodiment, samples that fail on the sequencer do not generate data files. These samples designated to be repeated do not continue with sequence analysis.

At this point, generation of individual sample FASTQ files may be performed. In an embodiment, the generation of CCS BAM files, demultiplexing and generation of FASTQ files is performed as disclosed in the Examples herein. Or, other methods may be used. Thus, in certain embodiments, preprocessing may comprise at least some of the steps of generating Circular Consensus Sequence (CCS) BAM files (402); merging the intermediate BAM files (404); demultiplexing using to generate individual BAM files corresponding to different barcode combinations (406); combining demultiplexed output by sample name and/or patient identifier (408); removing barcodes from sequences and generate individual sample FASTQ files (410); aligning sequences to barcodes and trimming the barcodes (412); converting BAM files to FASTQ files and copying FASTQ and CCS BAM files to final location (414); and triggering CLC Workflow (416).

The CLC Analysis workflow may be performed using the following steps. First, an NGS data analysis workflow may be executed on each sample using a current validated CLC Genomics Server version 418. Next, for each sample's FASTQ file the following steps may be performed. First, reads may be filtered to retain reads of 250-5000 bp length 420. Next, the reads are aligned to the SARS-CoV-2 reference genome (e.g., “NC_045512v2”) 422. This alignment may be performed using minimap2 to generate a BAM file. Or other alignment methods may be used. At this point, local realignment may be performed and variant calls made 424. This may be performed using the Low Frequency Variant Detection tool in CLC Genomics Server. Or, other methods may be used. At this point, both the assembly (BAM file) and detected variants (cf) are input into a downstream post-processing analysis 426. A script detects CLC process completion, initiating the launch of downstream analysis for samples in each SMRTcell.

Downstream Analysis

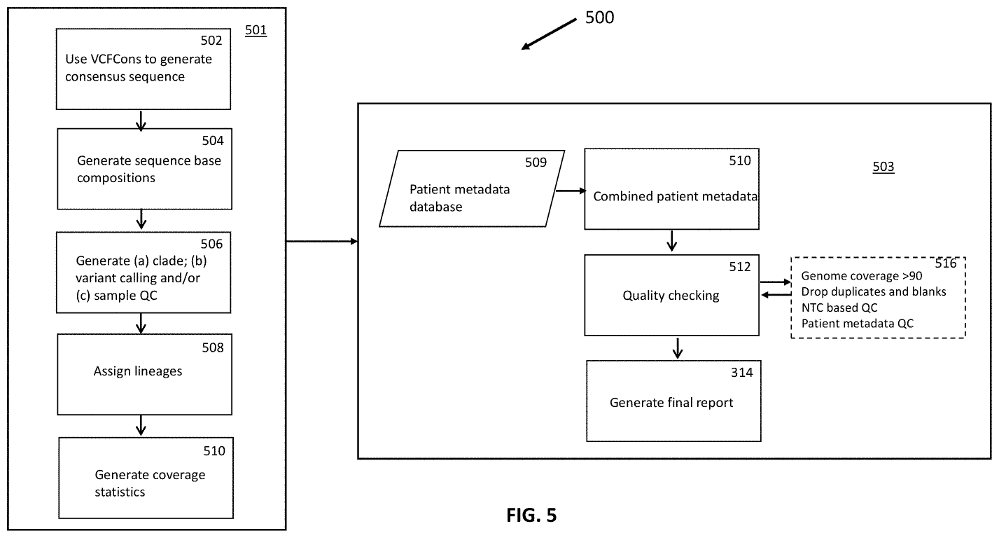

An example flow-chart for downstream (post-processing) analysis 500 is shown in FIG. 5. The steps for post-processing part 1 (501) may, in certain embodiments, be as follows. Using the appropriate reference file, VCFCons may be used to generate the consensus sequences based on sequence alignment and variant calls for each sample 502. For this analysis, a minimum coverage of 4 CCS reads and minimum alternate frequency of 0.5 may be used to assign a base to each genomic position. Or, a different threshold may be applied. In an embodiment, positions that do not satisfy this criterion are assigned an ambiguous base “N.” Next, sequence base compositions may be generated 504. In an embodiment, this may be used later to determine the percentage of non-ambiguous bases. In certain embodiments, this analysis may be performed with Seqtk or an alternate algorithm. At this point, any one or all of the following may optionally be generated using the consensus sequences as the input: (a) clade assignments; (b) mutation calling and (c) sample sequence quality check 506. In an embodiment, Nextclade is used for this analysis. Or, in certain embodiments, other algorithms may be used. At this point, lineages are assigned 508. In certain embodiments, Pangolin assigns lineages to the consensus sequence by generating the SARS-CoV-2 lineages, (known as the Pango nomenclature), then assigning a SARS-CoV-2 genome sequence lineage (Pango lineage). In an embodiment, Pangolin is set so as only to consider genomes that have at least 50% non-ambiguous bases. Finally, the coverage statistics may be generated 510. In certain embodiments, SummaryStat compiles results from Nextclade, Pangolin, and Seqtk and generates coverage statistics needed for later QC, including mean of median amplicon coverage and percent genome coverage. Or, another algorithm may be used for the compilation. In certain embodiments, for this analysis, the median coverage of the bases in 29 overlapping 1.2 kb regions that span the entire SARS-CoV-2 genome are calculated for each of the samples. Or, other thresholds may be used. Statistics of the distribution of these coverage values (minimum, 1st quantile, mean, median, 3rd quantile and maximum) may then be calculated for each sample. Also, the percent genome coverage may be calculated as the number of non-ambiguous bases (A, T, C, G) divided by the total sequence length, and lineage classifications are aggregated and only samples that produce a Nextclade result and Pangolin lineage call are retained for further processing.

At this point post-processing part 2 (503) may be initiated. Thus, again using the appropriate reference file strain surveillance-specific metadata 509, 510 (demographic data, percent genome coverage, and Ct values from the RT-PCR assay) QC is performed and the data added to the results 512. In an embodiment, samples that are missing metadata are dropped from the result set 516. Also, non-template QC is performed based on the no-template control (NTC) 516. Also, in certain embodiments, if the mean of the median coverage of the 29 genomic regions is >10 CCS reads, then all samples sequenced on the same plate are removed 516. Finally, coverage QC is performed 516. In an embodiment, samples with genome coverage >=90% are retained in the results. Also, in an embodiment, and samples with mean of median coverage >10 CCS reads were retained in the results. The results may then be transferred to a Report System location for generating patient reports with corresponding Pangolin lineages 514. In an embodiment, samples that failed to produce a result are reported as: no lineage was able to be determined. SARS-CoV-2 virus detected; no lineage information can be reported.

In certain embodiments, the lineage calling criteria may be as follows. Inclusion criteria: (1) CT <31; (2) corresponding metadata (strain surveillance); (3) >90% genome coverage; (4) mean of median coverage >10 CCS reads; (4) passing NTC control; and (5) Nextclade result and Pangolin lineage call. Exclusion criteria: (1) CT >31; (2) missing metadata (strain surveillance); (3) <90% genome coverage; (4) mean of median coverage <10 CCS reads; and (4) failing NTC control.

Revalidation

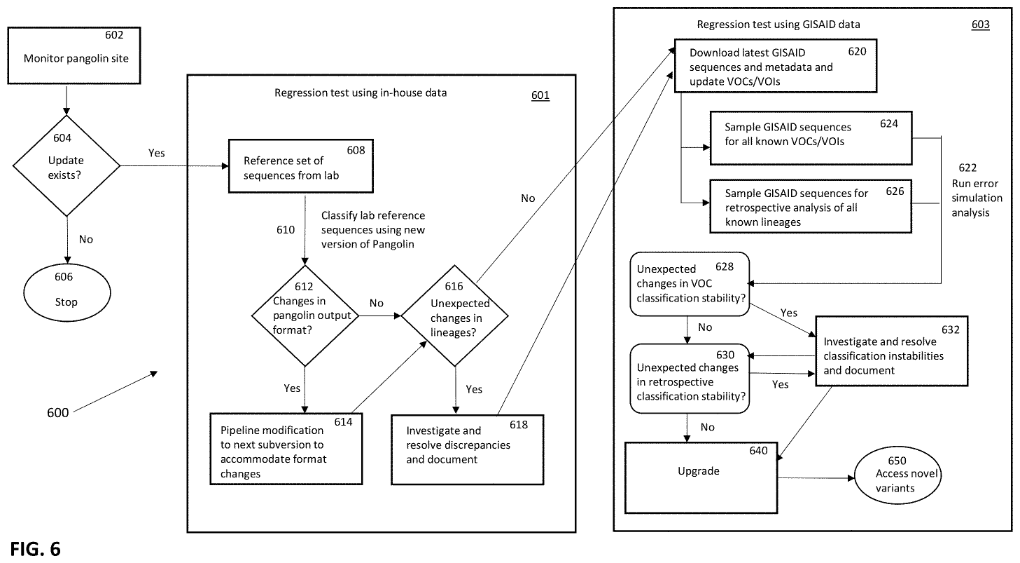

In certain embodiments the assay is revalidated in response to the emergence of new variants. In certain embodiments, at least some of these steps comprise computer-implemented steps for revalidation analysis. In certain embodiments, revalidating the classification accuracy of the Virseq assay 600 in response to the emergence of new variants (i.e. lineages) of the SARS-CoV-2 virus and concomitant changes to the pangolin classification software may be performed as depicted in FIG. 6 (see also Example 5). The analysis as depicted in FIG. 6 is developed for pangolin, but may be applied to other databases for phylogenetic assignment of viruses. The pangolin software is distributed through Dockerhub (at hub.docker.com/r/staphb/pangolin). Thus, in certain embodiments, the pangolin site may be monitored 602 and checked by downloading and installing an updated docker container at regular intervals (e.g., weekly, bi-weekly, monthly) for updates 604. In an embodiment, if there are no updates, no action is required 606.

If there are updates, a regression analysis may be performed using in-house laboratory data 601. In an embodiment, the new pangolin version may be used 610 to determine the lineage of in-house reference samples 608. The reference sample set 608 may include data from various sets (e.g., based on date, of accrual and/or COVID types). For example, data sets may be defined to be primarily Delta variants and/or Omicron lineages. Or, other types may be analyzed. In an embodiment, each sample in the reference set includes its consensus sequence as well as the history of its lineage classifications made by previous pangolin versions. The reference sample set 608 may be updated periodically to include samples representing newer, more prevalent lineages as pangolin versions are updated.

Next, the format of the pangolin software output may be compared with that of the previous version to determine if there are changes in the pangolin output format 612. If there are any changes these may be documented, and the laboratory pipeline modified to accommodate the change. The modified version may then be deployed to the QC environment for testing 614. Next, any changes in lineage calls may be assessed and compared with those expected from the software update change notes 616. For example, in certain embodiments, expected changes include reassignment among sublineages. If there are any unexpected changes in lineages (e.g., Delta sublineage changing to Alpha), these are investigated in detail and documented 618.

At this point, a second regression test may be performed using publicly available (GISAID) sequences and their metadata 603. Or other public databases may be used. For this analysis, the latest GISAID sequences may be downloaded and the metadata and pangolin lineages for all GISAID sequences obtained and the list of Variants of Concern (VOCs) (i.e., variants that are actively being tracked by the CDC and/or other health organizations) and Variants of Interest (VOIs) (i.e., variants being monitored by the CDC and/or other health organizations) updated based on WHO updates and the latest complete list of lineages 620. Next, a data simulator may be used to model the coverage and error properties of the in-house assay 622. In an embodiment, the simulator uses GISAID sequences as starting points and imposes simulated coverage and errors based on empirical coverage profiles and max-minor-allele frequencies from a collection of in-house samples. The resulting simulated samples are run through pangolin, and the lineage classifications are compared to those of the original GISAID sequences. Classification stability is defined as the rate at which mutated sequences maintain their expected lineage classifications. In an embodiment, two experiments in the regression are run to assess classification stability via simulation. Thus, the method may randomly sample up to 100 GISAID sequences for each VOC/VOI to assess the classification stability of these important lineages, regardless of their frequency in the sequencing data available 624. Or, more or fewer GISAID sequences for each VOC/VOI (e.g., 50, 200, 400, 500 or more) may be sampled depending on the needs of the analysis. This can allow for assessing classification stability of emerging variants as well as new sublineages of existing ones. Additionally, the method may randomly sample 10,000 GISAID sequences from the database for a frequency-based retrospective analysis of lineage classification stability 626. Or, more or fewer retrospective GISAID sequences may be sampled depending on the needs of the analysis. This may allow stability to be quantified relative to historical prevalence.

The output of the data simulator experiments is then reviewed, checking for unexpected changes in classification stabilities with respect to previous regression tests using GISAID data for the VOC/VOI data 628 and the retrospective data 630. In certain embodiments, any unexpected instabilities are investigated and documented 632. In certain embodiments, the upgrade is accepted upon satisfying certain parameters. In some cases, the upgrade is requested if the median VOC/VOI concordance between the simulated data and reference sequence is at least 90% 640. In cases where these criteria are not met, additional investigation may be needed.

In certain embodiments, if the new discordant lineage(s) is/are novel the samples may be tested for confirmation. If the discordant variant(s) is/are not novel variant(s), the method may include a further investigation to find the root cause of discordance. This can involve looking at the coverage of the reference sequence as well as the simulated sequences to ensure that it is not an undesirable drop in base coverage in specific regions. Additionally, and/or alternatively this may involve rerunning the simulation with another seed to see if this discordance is reproduced. If it is, the upgrade may be halted.

At this point the novel variants may be assessed using the methods and systems disclosed herein 650. For successful surveillance of emerging variants (lineages), it may be helpful to review the potential impact on the molecular loop inversion probe amplification by conducting an in silico analysis. Thus, the method may further include identifying the location of the individual sequence variants in the emerging lineages and the associated molecular loop probes to assess the potential for interference in probe binding. In an embodiment, a conservative estimate that the novel sequence variant overlapping with any probe will impact hybridization is used. Additionally, and/or alternatively, adjacent probes in the region may be reviewed to ensure coverage of the novel sequence variant. For any sequence variant that could result in a reduction of coverage within a particular region, the impacted probes within the pangolin lineage update validation summary are documented.

### Systems for NGS SARS-CoV-2 Strain Determination

Also disclosed are systems for performing the methods herein. For example, the system may comprise a station or component (or stations or components) for performing various steps of the methods. In certain embodiments, a station or component may comprise a robotic or computer-controlled station or component for performing a step or steps of the method. In certain embodiments, disclosed is a system for performing at least some of the steps of: (a) identifying a sample from a subject as positive for SARS-CoV-2 nucleic acid and/or antibodies to SARS-CoV-2; (b) generating a sample-specific SARS-CoV-2 nucleic acid from the sample; (c) performing nucleic acid sequencing on the sample-specific SARS-CoV-2 nucleic acid; and (d) determining whether the nucleic acid sequence comprises a SARS-CoV-2 variant sequence.

Thus, the system may comprise a station or component for obtaining samples for testing. The samples may be those for which the COVID status is not known, or samples that have previously tested positive for COVID. In certain embodiments, the positive samples may be identified using an approved EUA approved COVID-19 RT-PCR Test (e.g., Labcorp EUA200011 and/or EUA203057). In this way, results are for the identification of the SARS-CoV-2 strain infecting an individual after detection of viral RNA in the sample.

In certain embodiments, the system may comprise a station or component for performing the step of generating a sample-specific SARS-CoV-2 nucleic acid comprises using reverse transcriptase polymerase chain reaction (RT-PCR) to generate a sample-specific SARS-CoV-2 cDNA. The system may also comprise a station or component for hybridizing one strand of the sample SARS-CoV-2 cDNA to a single-stranded probe DNA template comprising a pair of SARS-CoV-2 probes, wherein the first probe is positioned at the 3′ end of the probe DNA template and the second probe is positioned at the 5′ end of the probe DNA template. In certain embodiments, the probe sequences are selected as tiled probes that bind at spaced intervals along a SARS-CoV-2 genome. For example, in alternate embodiments, the probes may be spaced by about 100, or 200, or 300, or 400, or 500, or 600, or 700, or 800, or 900 or more than 1,000 base pairs. Or, spacings within this range (e.g., 450, 550, 650 or 750) may be used. The probes may be tiled across greater than 99% (e.g., 99.6%) of the 30 kb SARS-CoV-2 viral genome. Also, in certain embodiments, the single-stranded probe DNA template further comprises universal sequencing primers (e.g., M13 primers) positioned internal to the probe sequences. Additionally, the single-stranded probe DNA template may further comprise an adaptor sequence for the addition of a barcode sequence used to correlate the SARS-CoV-2 sample-specific nucleic acid to a sample number. Also, the system may comprise a station and/or components for filling in the sequence between the two probes to generate a circular single-stranded probe DNA template comprising sequence specific to the sample SARS-CoV-2 cDNA between the two probe sequences and then releasing the circular single-stranded probe DNA template comprising sequence specific to the sample SARS-CoV-2 cDNA from the sample-specific SARS-CoV-2 DNA and digestion of the circular single-stranded probe DNA template comprising sequence specific to the sample SARS-CoV-2 cDNA to generate a linear DNA used as a template for nucleic acid sequencing. In certain embodiments, the system may comprise a station and/or components for modifying the linear probe DNA template to add adaptors and then amplifying the linear DNA template for DNA sequencing. In certain embodiments, the step of enrichment comprises purification step (e.g., bead purification).

The system may further comprise station(s) and/or components for DNA sequencing. In certain embodiments, the method employs whole genome sequencing. In certain embodiments, next generation sequencing (NGS) is used. Or, other types of sequencing such as but not limited to Sanger sequencing, shot gun sequencing, SMRT sequencing, pyrosequencing or nanopore sequencing. For example, in certain embodiments the PacBio whole genome sequencing with the corresponding SMRT link 9 software and analysis tools may be used.

The system may further comprise a station(s) and/or component(s) for data analysis. Thus, the system may comprise a station(s) and/or component(s) for determining whether the nucleic acid sequence comprises a SARS-CoV-2 variant sequence by aligning the sample SAR-CoV-2 sequence to a SARS-CoV-2 reference genome to generate a sample-specific assembly and consensus sequence and/or assessing the lineage for the sample. In certain embodiments, the system may include a station(s) and/or component(s) for identifying the geographic location of the subject.

Additionally, as disclosed herein, in certain embodiments, system may include a station(s) and/or component(s) may include uploading the results of the step of determining whether the nucleic acid sequence comprises a SARS-CoV-2 variant sequence into a depository for further classification if a variant is detected. The depository may be a CDC database. Or, other public depositories may be used.

As disclosed herein system may include a station(s) and/or component(s) for determining if an update to the depository has been made prior to the step of determining whether the nucleic acid sequence comprises a SARS-CoV-2 variant sequence.

The system may include station(s) and/or component(s) for automating various steps in the procedure. In certain embodiments, Hamilton Star robots may be used for sample plate setup. Additionally and/or alternatively, Formulatrix Mantis Liquid Handlers or other automated devices may be used for mastermix distribution.

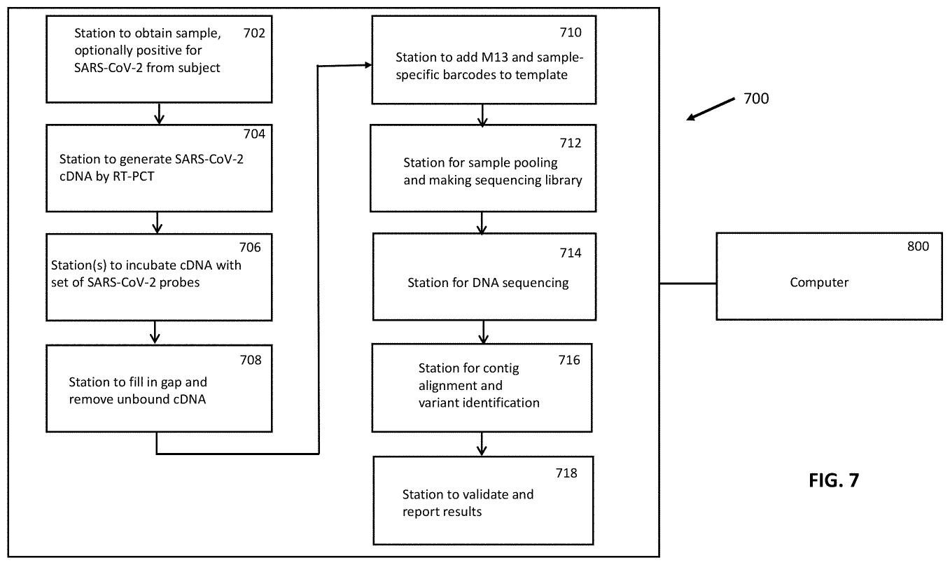

FIG. 7 illustrates an embodiment of a system 700 for performing any of the method steps of the disclosure. As illustrated in FIG. 7, the system may comprise a station or component for obtaining a sample for testing 702. In certain embodiments, the sample is positive for SARS-CoV-2. In an embodiment, the system comprises a station of a component to generate SARS-CoV-2 cDNA sequences by RT-PCT 704. The system may further comprise a station or component to incubate the SARS-CoV-2 cDNA with a set of tiled probes 706. In certain embodiments, the tiled probes are relatively evenly spaced across the SARS-CoV-2 genome. For example, in alternate embodiments, the probes may be spaced by about 100, or 200, or 300, or 400, or 500, or 600, or 700, or 800, or 900 or more than 1,000 base pairs. Or, spacings within this range (e.g., 450, 550, 650 or 750) may be used. The probes may be tiled across greater than 99% (e.g., 99.6%) of the 30 kb SARS-CoV-2 viral genome. After binding, the region in-between the two probes may be filled in with DNA polymerase and ligated to form a closed molecule. This may occur at the same station as the steps of incubating with tiled probes or at a different station and using different components 708. Non-binding or incomplete loops remain linear molecules and may be removed with exonuclease digestion. This may occur at the same station as the steps of incubating with tiled probes or at a different station and using different components. The system may further comprise a station for release of the circular molecules from the template cDNA and enrichment via amplification with a 3′ M13 universal sequence and 5′ sample specific barcodes 710. The system may further comprise a station and/or components for pooling samples and library generation 712.

The system may further comprise a station and/or components for sequencing the DNA 714 as well as a station(s) and/or component(s) for contig alignment and variant identification 716 using the methods disclosed herein. Also, the system may comprise a station(s) and/or component(s) to validate and report the results 718 as disclosed herein.

As illustrated herein, any of the method steps, stations or components may be automated, robotically controlled, and/or controlled at least in part by a computer 800 and/or programmable software. Thus, the system may comprise a computer-program product tangibly embodied in a non-transitory machine-readable storage medium, including instructions configured to run the system or any part (e.g., station or component) of the system and/or perform a step or steps of the methods of any of the disclosed embodiments. In some embodiments, a system is provided that includes one or more data processors and a non-transitory computer readable storage medium containing instructions which, when executed on the one or more data processors, cause the one or more data processors to perform part or all of one or more methods or processes disclosed herein and/or run any of the parts of the systems disclosed herein.

For example, disclosed is a system comprising one or more data processors, and a non-transitory computer readable storage medium containing instructions which, when executed on the one or more data processors, cause the one or more data processors to perform actions to direct at least one of the steps of: (a) identifying a sample from a subject as positive for SARS-CoV-2 nucleic acid and/or antibodies to SARS-CoV-2; (b) generating a sample-specific SARS-CoV-2 nucleic acid from the sample; (c) performing nucleic acid sequencing on the sample-specific SARS-CoV-2 nucleic acid; and (d) determining whether the nucleic acid sequence comprises a SARS-CoV-2 variant sequence.

Also disclosed is a computer-program product tangibly embodied in a non-transitory machine-readable storage medium, including instructions configured to run the systems and/or perform a step or steps of the methods of any of the disclosed embodiments. For example, in certain embodiments, the computer-program product tangibly embodied in a non-transitory machine-readable storage medium includes instructions configured to cause one or more data processors to perform actions to direct at least one of the steps of: (a) identifying a sample from a subject as positive for SARS-CoV-2 nucleic acid and/or antibodies to SARS-CoV-2; (b) generating a sample-specific SARS-CoV-2 nucleic acid from the sample; (c) performing nucleic acid sequencing on the sample-specific SARS-CoV-2 nucleic acid; and (d) determining whether the nucleic acid sequence comprises a SARS-CoV-2 variant sequence. Additionally and/or alternatively, in certain embodiments, the computer-program product tangibly embodied in a non-transitory machine-readable storage medium includes instructions configured to cause one or more data processors to perform actions to direct at least one of the components and/or stations of the system for performing actions to direct at least one of the steps of: (a) identifying a sample from a subject as positive for SARS-CoV-2 nucleic acid and/or antibodies to SARS-CoV-2; (b) generating a sample-specific SARS-CoV-2 nucleic acid from the sample; (c) performing nucleic acid sequencing on the sample-specific SARS-CoV-2 nucleic acid; and (d) determining whether the nucleic acid sequence comprises a SARS-CoV-2 variant sequence

The systems and computer products may perform any of the methods disclosed herein. One or more embodiments described herein can be implemented using programmatic modules, engines, or components. A programmatic module, engine, or component can include a program, a sub-routine, a portion of a program, a software component, or a hardware component capable of performing one or more stated tasks or functions. As used herein, a module or component can exist on a hardware component independently of other modules or components. Alternatively, a module or component can be a shared element or process of other modules, programs or machines.

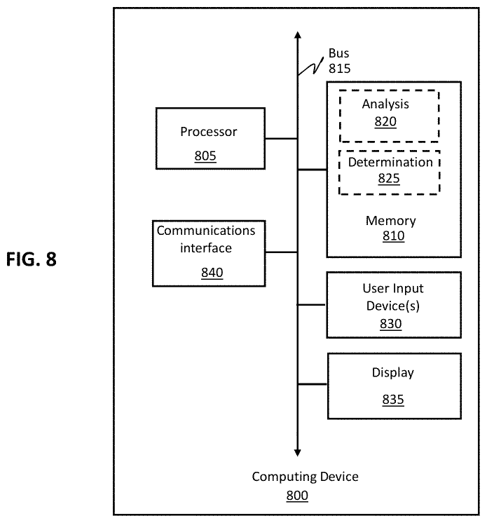

FIG. 8 shows a block diagram of an analysis system 800 used for detection and/or quantification of a pathogen. As illustrated in FIG. 8, modules, engines, or components (e.g., program, code, or instructions) executable by one or more processors may be used to implement the various subsystems of an analyzer system according to various embodiments. The modules, engines, or components may be stored on a non-transitory computer medium. As needed, one or more of the modules, engines, or components may be loaded into system memory (e.g., RAM) and executed by one or more processors of the analyzer system. In the example depicted in FIG. 8, modules, engines, or components are shown for implementing the methods of the disclosure.

Thus, FIG. 8 illustrates an example of a computing device 800 suitable for use with systems and methods according to this disclosure. The example of a computing device 800 includes a processor 805, which is in communication with the memory 810 and other components of the computing device 800 using one or more communications buses 815. The processor 805 is configured to execute processor-executable instructions stored in the memory 810 to perform one or more methods or operate one or more stations or components for detecting pathogen levels according to different examples, such as those illustrated in FIGS. 1-7 or disclosed elsewhere herein. In this example, the memory 810 may store processor-executable instructions 825 that can analyze 820 results for sample or test unit confirmation as discussed herein.

The computing device 800 in this example may also include one or more user input devices 830, such as a keyboard, mouse, touchscreen, microphone, etc., to accept user input. The computing device 800 may also include a display 835 to provide visual output to a user, such as a user interface. The computing device 800 may also include a communications interface 840. In some examples, the communications interface 840 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

### EXAMPLES

Certain embodiments of the method and systems of the disclosure are provided in more detail in the following Examples herein.

### Example 1

### Overall Method and Analysis of Results

Using next generation sequencing (NGS), surveillance testing can be performed on large numbers of samples and to generate an adequate number of viral genomes to track mutations and variants of concern as they arise. The overall test principle is as follows. First, cDNA is prepared from viral RNA using random priming for first strand synthesis. Next, inversion probes are annealed to target during a 16-hour hybridization. Next, gaps are filled in via polymerization and ligation. Next, non-reacted linear probes are removed and probe is released from target DNA. Next, captured target is enriched by PCR amplification using asymmetric barcodes. Next, PCR products are pooled, quantified, and SMRTbell hairpin adapters are ligated to amplicons and sequenced on the Pacific Biosciences Sequel II using a 15 hr movie.

At this point, lineage calls are made based on processing of the NGS sequence data. For this analysis, every condensed positive ‘cherry picked’ (discussed in more detail herein) includes: No Template Control (NTC) (i.e., molecular grade water) in well Al of the 96-well condensed positive plate. NTC results are reviewed prior to generation of the result file for a given SMRTcell. If an NTC is found to be invalid, results for all patient samples on the affected plate are not reported. Upon completion of processing of the NGS results for a given sequence cell, a result file is generated and saved. At this point, PacBio SMRTLNK software and custom molecular loop processing scripts are used to generate the FASTQ files for each of the samples. FASTQ results are analyzed using a genome analysis pipeline implemented in CLC genomics server version 6.5.6. This workflow starts with a sample-level fastq file, trims the primers and then uses Minimap2 to align to the SARS-COVID19 reference genome (“NC_045512v2”) to generate a bam file of alignment. After coverage checking, the bam file is used as the input for calling variants and for generating a sample-specific genome assembly. A consensus sequence for each sample is generated using “VCFcons” requiring a coverage of 4 CCS reads and alternate allele frequency of 50% at each base. The lineages for individual samples are assigned using the Pangolin package.

### Example 2

### Methods and Validation for VirSeq SARS-CoV-2 NGS Strain Determination

Genomic sequencing of SARS-CoV-2, the virus that causes COVID-19, can determine the specific strain of SARS-CoV-2. The strain information can potentially provide valuable information to clinicians and epidemiologist to aid in the public health response to the virus or future clinical treatments. The determination of a given strain is based on a combination of multiple variations in the genome detected from comparison of DNA sequencing results to the original Wuhan reference strain. This approach allows the identification of any new and emerging strains of SARS-CoV-2 as the virus changes over time without revalidation. The intended use of this assay is to result SARS-CoV-2 lineage, or strain, calls with samples that yield at least 90% genome coverage.

The overall test principle is as follows. Residual total nucleic acid extract from residual SARS-CoV-2 NAA diagnostic testing positive samples was cherry picked from run plates into a condensed positive plate using Hamilton STARs, and aliquoted into a sequencing run plate of 96, with 8 plates or 768 specimens in one production batch. A Molecular Loop Viral RNA Target Capture on PacBio was then used to process samples until PacBio sequencing. First, a Loop kit specific Thermo Fisher VILO reverse transcriptase was used to synthesize cDNA from RNA. The SARS-CoV-2 cDNA was then used as a target to anneal molecular loop probes as outlined in Table 1. Molecular loop probes were tiled across the full 30 KB SARS-CoV-2 genome and comprise two binding sites approximately 600 bp apart.

TABLE 1

| | Incubation | Incubation | |
|---|---|---|---|
| Step | Temperature ° C. | Time | |
| 1 | 25 | 10 | min |
| 2 | 50 | 50 | min |
| 3 | 95 | 1 | min |
| 4 | 55 | 16-24 | hours |

After binding, the approximately 600 bp regions in-between the two probes were synthesized with DNA polymerase and ligated to form a closed molecule using the hybridization conditions in Table 1 for an additional 60 minutes. Non-binding or incomplete loops remain linear molecules and were removed with exonuclease digestion (i.e., sample clean-up). Incubation times for clean-up are shown in Table 2. Samples were stored at −20° C. if not being used within about 2 hours for the next step.

TABLE 2

| | Incubation | Incubation | |
|---|---|---|---|
| Step | Temperature ° C. | Time | |
| 1 | 45 | 1 | hr |
| 2 | 95 | 3 | min |

| | | |
|---|---|---|
| 3 | 4 | Hold |

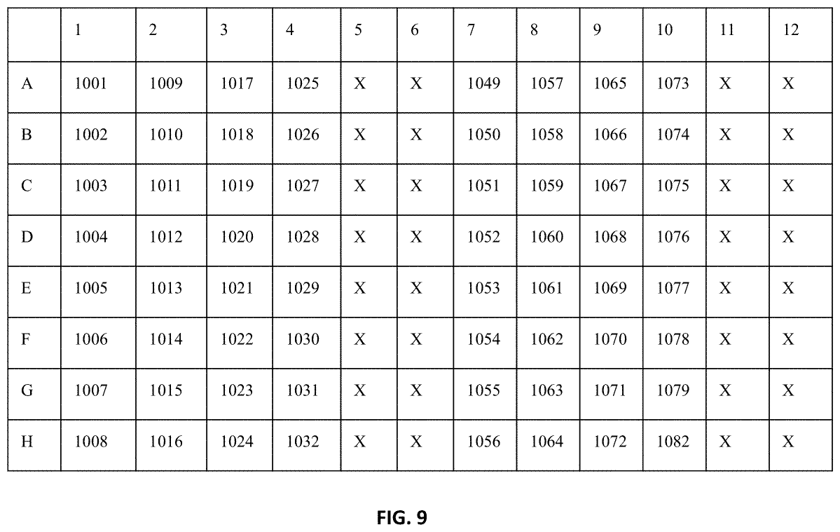

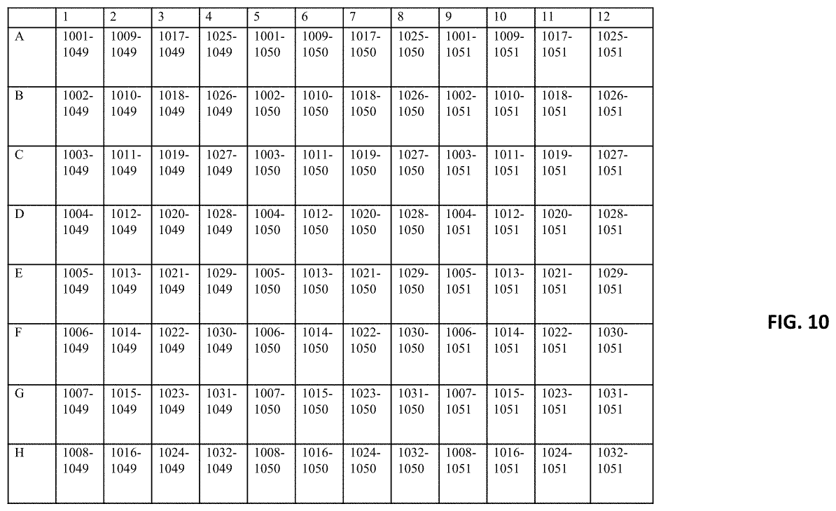

The resulting circular molecules (containing sample specific SARS-CoV-2 nucleic acid inserted between the two probe sites) were then released from the template cDNA and PCR amplified with sample specific barcodes. Conditions used for PCR amplification are shown in Table 3. Seven hundred and sixty-eight (768) asymmetric barcode combinations are needed to process one batch (i.e., 768 samples and controls). To do this, a plate of M13 barcoded primers was prepared (FIG. 9) and then ten 96-well plates were created by adding 20 μL of M13 forward barcoded primer and 30 μL of M13 reverse barcoded primer (FIG. 10). As shown in FIG. 9, columns 1-4 were M13 forward primers tailed with barcode 1001-1032 and columns 7-10 were M13 reverse primers tailed with barcode 1049-1079 and 1082. Each of the 32 forward primers were combined with different reverse primers to create asymmetrically barcoded pairs as shown for one example plate in FIG. 10.

TABLE 3

| | | Incubation | | |
|---|---|---|---|---|
| Step | Temperature ° C. | Time | | Cycles |
| 1 | 95 | 3 | min | 1 |
| 2 | 98 | 15 | sec | 23 |
| 3 | 55 | 15 | sec | |
| 4 | 72 | 90 | sec | |

| | | | |
|---|---|---|---|
| 5 | 4 | Hold | 1 |

Next, samples were pooled (or stored at −20° C. until pooling was performed). For pooling, 8 reaction plates were retrieved from storage, spun down, and an aliquot (e.g., 5 μL) of each reaction was transferred into an 8 mL tube. Generally, 768 samples plus controls were pooled prior to sequencing.

At this point samples were purified using bead purification with AMPure PB beads. Using 500 μL of the pool, AMPure PB Bead (0.70×) cleanup was performed by adding 350 μL of PB AMPure beads mixing, centrifuging to pellet the beads, incubating 5 min at room temperature, and centrifugation and magnetic separation to collect the beads. The supernatant was removed, the beads washed with 80% ethanol, and the DNA eluted from the beads with elution buffer and quantitated.

At this point the SMRTbell library was prepared using 1000 ng of the pooled DNA. The pooled DNA was mixed with buffer (DNA Prep Buffer), NAD, DNA Damage Repair Mix v.2, and incubated at 37° C. for 30 minutes. After returning to 4° C., end repair was performed by the addition of End Prep Mix, Reaction Mix 1 and incubating at 20° C. for 30 minutes, at 65° C. for 30 minutes, then returning the reaction to 4° C. At this point adapters were added using Reaction Mix 2, Overhand Adapter v3, Ligation Mix, Ligation Additive and Ligation Enhancer and the samples incubated at 20° C. for 60 minutes to ligate the probe construct, at 65° C. for 10 minutes to inactivate the ligase, then returned to 4° C. Enzyme clean-up was then performed and the sample purified with AMPure (0.6×) beads above using 100 uL elution buffer. The AMPure bead clean-up was repeated using a smaller volume (20 uL) elution buffer and the DNA quantitated.

Samples were sequenced on a PacBio Sequel II. Each 96-well plate in the batch of ten requires a unique set of asymmetric barcodes. FIG. 10 shows one example map for one plate. Other combinations were used for other plates such that 10 plates with unique combinations were used. For example, in a second plate wells A1-H4 would be a combination of M13 forward primers 1001-1032 with M13 reverse primer 1052, wells A5-H8 would be a combination of M13 forward primers 1001-1032 with M13 reverse primer 1053, and wells A9-H12 would be a combination of M13 forward primers 1001-1032 with M13 reverse primer 1054 and so forth for additional plates.

After sequencing, PacBio SMRTLNK software and custom molecular loop processing scripts were used to generate the FASTQ files for each sample. FASTQs were analyzed using a genome analysis pipeline implemented in the CLC genomics server version 6.5.6. This workflow started with a sample-level fastq file, primers were trimmed, and Minimap2 was used to align to the SARS-COVID19 reference genome (“NC_045512v2”) to generate a bam file of alignment. After coverage checking, this bam file was used as the input for calling variants and for generating a sample-specific genome assembly. A consensus sequence for each sample was generated using “VCFcons” requiring a coverage of 4 CCS reads and alternate allele frequency of 50% at each base. The lineages for individual samples were then assigned using the Pangolin package and resulted.

The following controls were included. A No Template Control (NTC) was included on each plate on a run for all steps to verify that there was no contamination across samples and reagents. This control was analyzed by sequencing. A failed NTC was a sample that produced a strain call with 90% genome coverage. A positive control was included on each plate of a run. For validation, a previously run sample was used as a positive control. Metrics to determine if a sample passed or failed included percent genome coverage, minimum depths of coverage, and resolution of strain lineage call.

Specimen requirements were as follows. Extracted Nucleic Acid derived from a sample with a positive result from an EUA approved SARS-CoV-2, NAA test with a CT of less than 26 for ˜90% success rate. Higher CTs or no-CT metadata samples were deemed to be acceptable but increased risk of inability to report a result.

Acceptable result metrics were as follows: >90% genome coverage and a mean of median read coverage >10 CCS reads.

A. Results

Precision (Repeatability): Intra-Assay

Intra-assay repeatability was assessed on 3 replicates of 11 nucleic acid samples of various assumed typings from current SARS-CoV-2 CDC surveillance testing. Samples ranged in CT value and a wide range of read counts in the original run. Further, samples were diluted 1:4 to allow ample total nucleic acid input to all intra and inter-assay experiments. The Acceptance Criteria was defined as ≥95% repeatability for all strains reaching a reporting threshold of ≥90% coverage of the SARS-CoV-2 genome.

The strain call, percent genome coverage (displayed in percent missing), and read count was compared (Table 4). All eleven sample's strain call was 100% concordant across the three replicates with all replicates meeting 90% genome coverage and ample read depth. Acceptance criteria of 95% accuracy of strains with 90% genome coverage was met.

TABLE 4

| | | Expected | | | |
|---|---|---|---|---|---|
| Specimen # | Metric | Lineage | PBT5073A | PBT5073B | PBT5073C |
| 1522917599 | lineage | P.1 | P.1 | P.1 | P.1 |
| | percent missing | | 0.74 | 0.85 | 0.84 |
| | Avg Read depth | | 58.1 | 96.83 | 52.34 |
| 1523600378 | lineage | B.1.628 | B.1.628 | B.1.628 | B.1.628 |
| | percent missing | | 0.41 | 0.41 | 0.41 |
| | Avg Read depth | | 176.36 | 156.59 | 125.76 |
| 1523600378 | lineage | A.2.5 | A.2.5 | A.2.5 | A.2.5 |
| | percent missing | | 0.74 | 0.63 | 0.52 |
| | Avg Read depth | | 211.69 | 284.09 | 309.79 |
| 1535487742 | lineage | B.1.1.7 | B.1.1.7 | B.1.1.7 | B.1.1.7 |
| | percent missing | | 0.52 | 0.84 | 0.74 |
| | Avg Read depth | | 93.86 | 48.53 | 86.66 |
| 1537276386 | lineage | B.1.526 | B.1.526 | B.1.526 | B.1.526 |
| | percent missing | | 1.45 | 1.13 | 0.78 |
| | Avg Read depth | | 31.22 | 50.91 | 52.21 |
| 1538337948 | lineage | C.37 | C.37 | C.37 | C.37 |
| | percent missing | | 0.53 | 0.63 | 1.34 |
| | Avg Read depth | | 138.38 | 133.74 | 57.17 |
| 1538338001 | lineage | B.1.1.7 | B.1.1.7 | B.1.1.7 | B.1.1.7 |
| | percent missing | | 1.04 | 0.53 | 2.16 |
| | Avg Read depth | | 30.41 | 51.59 | 13.55 |
| 1544492013 | lineage | B.1.1.7 | B.1.1.7 | B.1.1.7 | B.1.1.7 |
| | percent missing | | 0.41 | 0.41 | 0.53 |
| | Avg Read depth | | 177.12 | 294.67 | 148.76 |
| 1562914144 | lineage | P.1 | P.1 | P.1 | P.1 |
| | percent missing | | 0.63 | 0.74 | 0.73 |
| | Avg Read depth | | 338.03 | 207.5 | 100.93 |
| 1568334279 | lineage | B.1.617.2 | B.1.617.2 | B.1.617.2 | B.1.617.2 |
| | percent missing | | 1.00 | 0.74 | 1.54 |
| | Avg Read depth | | 27.9 | 46.84 | 25.21 |
| 1583805067 | lineage | B.1.526 | B.1.526 | B.1.526 | B.1.526 |
| | percent missing | | 1.25 | 1.52 | 0.67 |
| | Avg Read depth | | 22.86 | 17.16 | 28.72 |

Precision (Reproducibility): Inter-Assay

Inter-assay repeatability was assessed on 3 replicates of ten nucleic acid samples of various assumed typings from current SARS-COV-2 CDC surveillance. Samples were identical to ones used in intra-assay experiments with one sample being dropped from unintentionally being excluded from the final run. Samples ranged in CT value and a wide range of read counts in the original run. Further, samples were diluted 1:4 to allow ample total nucleic acid input to all intra and inter-assay experiments. The Acceptance Criteria was defined as ≥95% repeatability for all strains reaching a reporting threshold of ≥90% coverage of the SARS-CoV-2 genome.

The strain call, percent genome coverage (displayed in percent missing), and read count was compared (Table 5). For all 10 replicates there were no discordant results. Nine samples produced expected linage calls across triplicates. One sample, purposely chosen for borderline read coverage, failed to produce a result every time due to lack of genome coverage. Overall, 93% of samples produced an identical strain typing, and 100% of samples released accurate results meeting acceptance criteria.

TABLE 5

| Specimen | Metric | PBT5073C | PBT5075 | PBT5080 |
|---|---|---|---|---|
| 1522917599 | lineage | P.1 | P.1 | P.1 |
| | percent missing | 0.84 | 0.96 | 0.84 |
| | Avg Read depth | 52.34 | 34.9 | 17.06 |
| 1523600378 | lineage | B.1.628 | B.1.628 | B.1.628 |
| | percent missing | 0.41 | 0.41 | 0.41 |
| | Avg Read depth | 125.76 | 134.41 | 130.71 |
| 1523600378 | lineage | A.2.5 | A.2.5 | A.2.5 |
| | percent missing | 0.52 | 0.57 | 0.32 |
| | Avg Read depth | 309.79 | 219.86 | 588.37 |
| 1537276386 | lineage | B.1.526 | B.1.526 | B.1.526 |
| | percent missing | 0.78 | 1.32 | 0.41 |
| | Avg Read depth | 52.21 | 43.03 | 39.19 |
| 1538337948 | lineage | C.37 | C.37 | C.37 |
| | percent missing | 1.34 | 1.13 | 0.42 |
| | Avg Read depth | 57.17 | 60.21 | 91.09 |
| 1538338001 | lineage | B.1.1.7 | B.1.1.7 | B.1.1.7 |
| | percent missing | 2.16 | 23.61 | 0.73 |
| | Avg Read depth | 13.55 | 7.38 | 13.88 |
| 1544492013 | lineage | B.1.1.7 | B.1.1.7 | B.1.1.7 |
| | percent missing | 0.53 | 0.41 | 0.41 |
| | Avg Read depth | 148.76 | 226.22 | 309.86 |
| 1562914144 | lineage | P.1 | P.1 | P.1.7 |
| | percent missing | 0.73 | 0.64 | 0.73 |
| | Avg Read depth | 100.93 | 318.59 | 77.97 |
| 1568334279 | lineage | B.1.617.2 | B.1.617.2 | B.1.617.2 |
| | percent missing | 1.54 | 1.97 | 0.52 |
| | Avg Read depth | 25.21 | 17.31 | 19.99 |
| 1583805067 | lineage | B.1.526 | None | B.1 |
| | percent missing | 0.67 | 67.10 | 9.82 |
| | Avg Read depth | 28.72 | 3.03 | 2.86 |

Concordance

The relative accuracy was established by direct comparison of results with those generated by alternate methods. There were two methods used for comparison of positives, Illumina Sequencing and Amplicon Pacbio Sequencing. While Pacbio is the validated sequencing technology, the Molecular Loop method is mechanistically distinct in pre-sequencing steps from traditional amplicon sequencing. Negatives were sequenced on Illumina only.

The individual comparison studies used are listed below.

Illumina Artic Sequencing
93 Negative NAA samples
72 SARS-CoV-2 samples sequenced in winter 2020
29 samples previously sequenced at CMBP on Molecular loop
50 samples previously sequenced at DNA Identification on Molecular loop.

Pacbio Amplicon Sequencing
122 samples amplicon sequenced at 90% coverage and ran in duplicate on Molecular loop.

To set up a baseline for minimum read coverage, 110 NTC's from the validation runs and current RUO strain surveillance ran during the validation timeline were used to set a minimum read coverage threshold set at 4 CCS reads. For Illumina concordance, 93 Negatives were randomly chosen from a CMBP NAA diagnostic production run and re-extracted after initial testing to ensure adequate volume. Seventy-two samples of strains circulating in the winter of 2020 and sequenced in January 2021 were resequenced on Molecular Loop. Further, seventy-nine samples from CMBP and DNA were chosen for Illumina parallel testing based on initial strain call, CT and read coverage to ensure diversity. Three hundred and eighty-two samples originally Amplicon sequenced on Pacbio were reprocessed on Molecular Loop in duplicate. The duplicates varied only slightly in their composition of Thermo Fisher VILO RT master mix that was previously shown to be comparable. In the initial amplicon sequencing run 122 of 382 samples produced >90% genome coverage. Only samples with initial 90% coverage were used for further analysis of molecular loop results.

The Acceptance Criteria was ≥95% accuracy for all strains reaching a reporting threshold of ≥90% coverage of the SARS-CoV-2 genome.

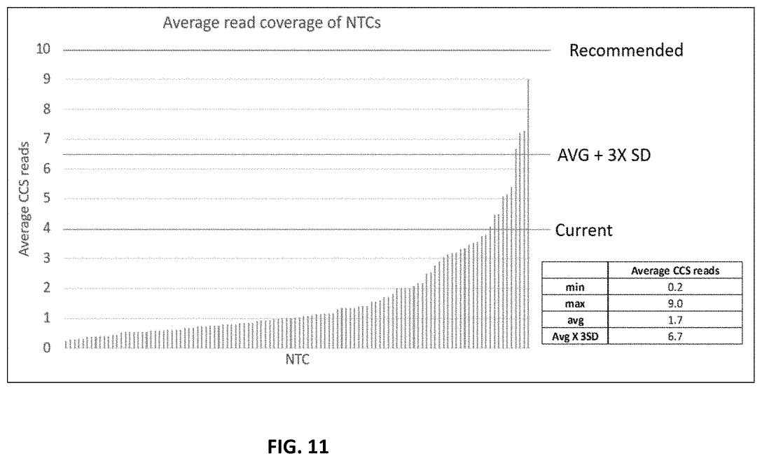

Read coverage threshold: Average, minimum and maximum mean of median amplicon coverage, here referred to as average read coverage, was analyzed for validation runs and productions runs. A distribution of average read coverage is shown in FIG. 11. The minimum, maximum and average were 0.24, 9 and 1.7 CCS reads respectively. Typical thresholds are set by 3× the standard deviation plus the average which for this analysis equaled 6.67. However, for rapid processing, the cutoff read coverage for a base pair to be used in strain typing was set at 10 CCS reads, or 10% plus the max NTC.

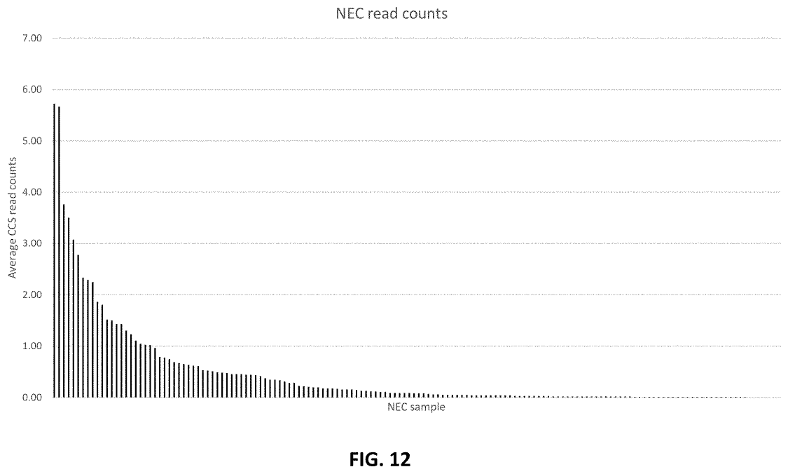

Illumina Artic Sequencing: 93 samples previously determined to be negative were sequenced in duplicate on Molecular Loop and on Illumina in parallel. There was 98.3% concordance between the two technologies with two samples resulting in reportable genomes on Illumina, and one on Molecular loop. Further investigation revealed both samples resulted on Illumina were indeed positive for nucleic acid amplification (NAA) and mistakenly included in the validation. The average read counts of the other 91 samples in duplicate further confirmed the conservative read depth threshold (FIG. 12).

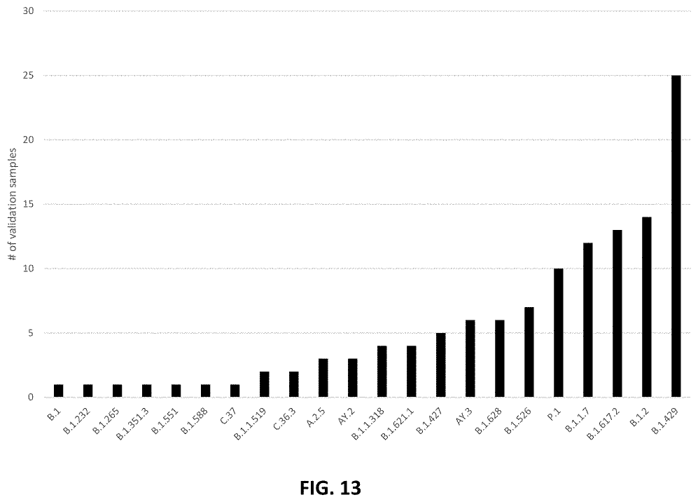

72 samples previously sequenced on Illumina from strains circulating in January 2021 were resequenced on Molecular loop. To represent current strains in circulation, 79 samples previously sequenced on Molecular loop at CMBP and DNA Identification were resequenced on Molecular loop and Illumina in parallel. After removal of samples damaged in transit between testing sites or failed the comparative sequencing reaction, 123 successfully produced strain calls on both platforms were analyzed (FIG. 13).

Of the 72 samples, 51 met QC thresholds of 10 CCS read depth and 90% genome coverage for a 71% success rate. This reportable genome rate was similar to CDC strain surveillance reportable genome rate at DNA of 72.5% during the month of the validation. All strain results were 100% concordant out of the 51 reportable results (Table 6). Inclusion of results with less than 10 CCS read depth on average resulted in 10/13 (77%) matching strain results and a total concordance of 61/64, 95.3%. Analysis of samples below 90% genome coverage only had 2/7 identical strain results.

TABLE 6
Winter 2020 Circulating Strain Accuracy

| QC Metrics | concordant | total | Accuracy |
|---|---|---|---|
| >90% coverage > 10 CCS reads | 51 | 51 | 100% |
| >90% coverage < 10 CCS reads | 10 | 13 | 77% |
| <90% coverage | 2 | 7 | 29% |

Sixty-six of 72 samples originally sequenced on Molecular Loop at CMBP and DNA were successfully sequenced after dilution with adequate read depth on Illumina and Molecular loop. All 72 samples produced 90% coverage and a strain typing of which 71 were able to identical strain calls to the Illumina reference method. Overall, all reportable results were 100% concordant between parallel technologies (Table 7).

TABLE 7
Summer 2021 Circulating Strain Accuracy

| QC Metrics | concordant | total | Accuracy |
|---|---|---|---|
| >90% coverage > 10 CCS reads | 66 | 66 | 100% |
| >90% coverage < 10 CCS reads | 6 | 6 | 100% |

Amplicon Pacbio Sequencing: Out of the 122 samples with 90% coverage on amplicon sequencing, 116 were repeated at 90% coverage for both replicates. There was 100% concordance between the 116 molecular loop replicate strain typings. Overall, parallel testing between molecular loop and traditional amplicon sequencing were 98.2% concordant.

Analytical Sensitivity/Specificity

Heat-inactivated SARS-CoV-2 strains B.1.1.7 (VR-3326HK™), Hong Kong/VM20001061 and Italy-INMI1 genomes are characterized by ATCC. For analytical sensitivity all variants were identified using the analysis pipeline and compare to the published ATCC strain variant datasets. Traditionally in human genome sequencing a variant of interest is analyzed and validated and a False Discovery Rate (FDR), which normalizes false positives (FP) to all positive calls (FP+TP where TP=true positive) rather than to all negatives. However, with Sars-Cov-2 there is a combination of a 4 to 20+ variants at defined positions for a given strain that lead to the strain call. Also, due to complete genome sequencing of viral RNA, there are multiple highly repetitive regions known to cause variation in sequencing data that are not relevant to current strain typings. Strain typing programs such as Nextclade can take this into account. Therefore, sensitivity was determined by the number of called variants documented for strain divided by the total variants. In addition to FDR, specificity was calculated by the total number of false variants called compared to accurately sequenced base pairs. The assembled genome was used as input in Pangolin which calls variants and outputs a strain typing. No variant calls, and only strains were output for further analysis. Therefore, sensitivity and specificity was calculated using variant calls from a separate genome variant caller, CLC, and Nextclade Sars-Cov-2 specific variant caller which takes into account repetitiveness and difficult to sequence viral regions when making a variant call. The Acceptance Criteria was as follows: (1) ≥90% analytical sensitivity with control RNA for variants in segments that are above minimum coverage; and (2) ≥90% analytical specificity with control RNA for variants in segments that are above minimum coverage. False Discovery Rate with control RNA for variants in segments that are above minimum coverage were documented but no acceptance criteria was set.

Both variant calling platforms were highly sensitive in their ability to detect variants with Nextclade at 96.23% and CLC at 98.11% sensitivity (Table 8). When comparing the overall specificity of determining a base pair across the genome both were >99.9% specific. However, the ability of Nextclade to adjust for repetitive and difficult to sequence regions was obvious by the number of false variants detected at 3, compared to CLC with no adjustment process at 23. This lead to a discrepancy in false discovery rates of variants with 5.36 for Nextclade and 30.26 for CLC indicating false variant discovery is in hard to sequence repetitive viral regions; these regions are not relevant to current strain typing. Together, all acceptance criteria were met and the Molecular Loop process is highly sensitive and overall specific, with high FDR from viral regions not analyzed in current strain typing algorithms.

TABLE 8
Sensitivity and specificity calculations of ATCC sequenced controls.

| | | | # | | | | | |
|---|---|---|---|---|---|---|---|---|
| Variant | ATCC | # called | Expected | # false | BP | | | |
| Analysis | strain | expected | variants | var | sequenced | Sensitivity | Specificity | FDR |
| Nextclade | b1117 | 37 | 37 | 2 | 29683 | | | |
| | HK | 11 | 13 | 0 | 29780 | | | |
| | ITLY | 3 | 3 | 1 | 29979 | | | |
| | Total | 51 | 53 | 3 | 89442 | 96.23% | 99.997% | 5.36% |
| CLC | b1117 | 37 | 37 | 8 | 29683 | | | |
| | HK | 12 | 13 | 9 | 29780 | | | |
| | ITLY | 3 | 3 | 6 | 29979 | | | |
| | Total | 52 | 53 | 23 | 89442 | 98.11% | 99.974% | 30.26% |

Assay Tolerance

The assay tolerance for nucleic acid input can be thought of as the tolerance to variation in the amount of analyte added to the reactions. While normally expressed in cp/μL, ˜80% of samples assayed will be from an EUA NAA SARS-CoV-2 test which provides each sample's corresponding cycle threshold (CT) value. As such, CT was used in place of cp/μL as the input metric for analysis and guidance. Sequencing viral genomes from residual NAA testing inherently has a high failure rate, which is directly related to the specimen's viral titer and RNA integrity and can vary dramatically between samples. While the failure rate is driven by RNA titer (CT), with a conservatively set background the increase in failures observed in higher CT samples will not lead to discrepant results, and only increase the cost of the assay. The aim of this validation's assay tolerance experiment was to set baselines for expected success rates at a given CT, but does not limit what samples are attempted to have genomes sequenced.

9,718 production results across 3 sites were analyzed for success rate to produce a result based on their nucleocapsid target #1 (N1) CT value. First, samples were binned by ability to produce a genome at 90%, and CT value at 1 integer increments rounded up to the nearest whole number. For example, 30.1 CT was calculated under the 31 bin. All samples with a CT of <16 were included in the 16 bin. All samples missing CT metadata were removed from analysis. The Acceptance Criteria were: (1) the manufacturer recommendation for 10,000 copies of RNA for sequencing with acceptable variation in input concentration used meet the following acceptance criteria for analysis; and a CT group of at least 20 samples

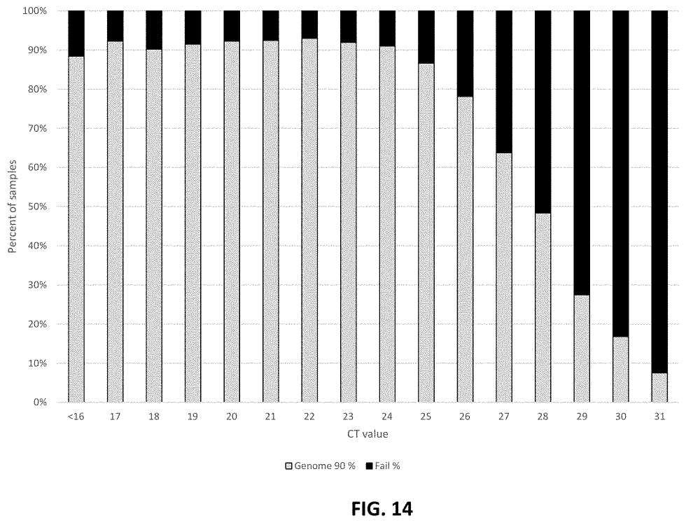

Over 8815/9718 production samples had the corresponding CT metadata. There was no deterioration in ability to generate ˜90% genome coverage from <16 to 24 CT analysis bins (FIG. 14). Starting at 25 CT, there was a precipitous decline in the ability of a sample at a given CT to generate a genome with 90% coverage, with 30.14→31 CT bin only reporting 7.65% samples. It is recommended that if a CT cutoff for assay input is required, that samples must be at <26 CT, which had a 78% genome yield rate.

Analyte Stability

Samples in this validation were stored for a minimum period of 4 weeks which exceeds the period of time over which the samples are tested in the clinical laboratory. Long-term stability should be determined by storing at least three aliquots under the same conditions as the study samples. The volume of samples should be sufficient for analysis on three separate occasions. The stability of the analyte in biological matrix at intended storage temperatures should be established.

The stability of the analyte under various storage conditions was established by measurement of concordance at various lengths of storage. After NAA diagnostic testing, extracted nucleic acid was shipped on dry ice to the testing laboratory and stored at −20° C. before sequencing. All samples used in validation were residual production samples and the stability experiments described below are in addition to the process of collecting and shipping samples to the sequencing laboratory. Analyte stability was measured in two separate experiments. In the first experiment, ten samples used in inter-assay precision were defrosted, assayed, and refrozen three times across a one-month time point. Samples represented various strains, CT values and original read coverage. In the second experiment, twelve samples comprising of Alpha, Beta and Delta Variants of Concerns (VOCs) with ranging original read sequence depth were resequenced after one month of −20° C. storage that entailed three freeze thaws. The Acceptance Criteria was defined as storage conditions were considered suitable if the sample yields the same strain detection after the defined length of storage and ≥90% accuracy for reportable strain results.

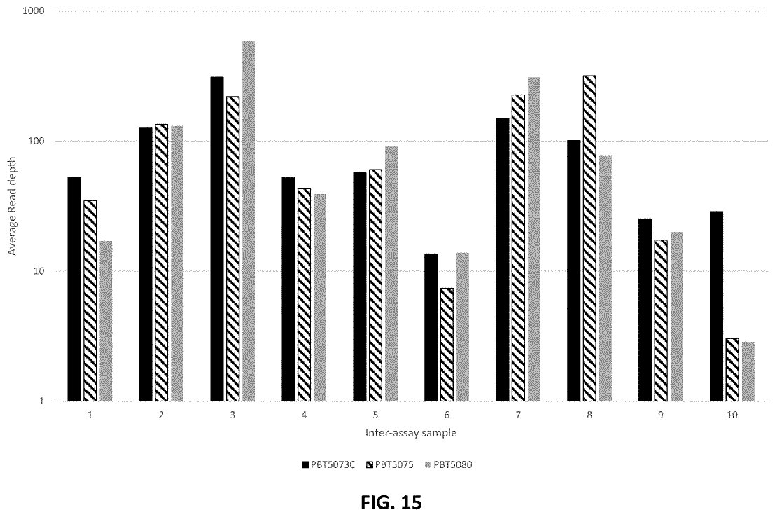

Inter-assay results used in stability study are found above in Table 5. Only two replicates of one sample, 1583805067, failed to produce 90% coverage and 10 CCS reads for 90% reproducibility. Further, there was no observed reduction in sample specific read count on the final stability time point (PBT5080) for three separate sequencing runs (PBT5073 PBT5075 and PBT5080) (FIG. 15).

Reprocessing VOCs had 9/12 samples produce identical strain results. One sample produced a AY.3 while the original results was 1.617.2. Both AY.3 and 1.617.2 comprise the Delta VOC and since the original Week 24 result are now classified as distinct sub-strains of the Delta VOC. Further investigation revealed that the 38 CLC variants between the two results are identical and strain typing was due to differences in Pangolin strain caller versions. One sample was concordant, but lacked sufficient read depth to report a result. The only true discordant was originally reported a B.1.1.7 and upon repeat was B.1.621.1. As both original and stability sequencing resulted in ample read coverage with minimal shared variants called between runs, it is believed the discrepancy may have resulted from sample switch. The overall accuracy was 91.6% with the discrepant result not attributed to stability issues.

### Example 3

### Cherry Picking

Hamilton MicroLab STAR liquid handlers are used to transfer specimens from source plates containing both positive and negative patient samples into condensed PCR plates containing only positive samples for sequencing. Informally, this process is referred to as “cherry picking”. Specimens are extracted total nucleic acid from positive specimens with a CT <31.

### Example 4

### Analysis of Sequencing Data for SARS-CoV-2 Strain Determination

The upstream analysis included monitoring the sequencer runs for completion, demultiplexing to generate individual sample FASTQ files, and triggering the alignment of each to the SARS-CoV-2 reference genome to generate alignments and variant calls. The downstream analysis for samples in each SMRTCell included generating all the results including the lineage classifications for each sample.

Upstream Analysis

An example flow-chart for upstream analysis is shown in FIG. 4. For the analysis invocation, PacBio/Molecular Loop raw data was deposited from the sequencer to the AWS drop directory. A script detected when a run completed file was created and copied the data to a ready-for-demultiplexing folder. Samples that failed on the sequencer did not generate data files. These samples were designated to be repeated not used for sequence analysis.

At this point, demultiplexing and generation of individual sample FASTQ files was performed using the following steps: (1) generation of Circular Consensus Sequence (CCS) BAM files using PacBio's SMRTLINK CCS program; (2) merging the intermediate BAM files using samtools; (3) demultiplexing using the PacBio lima program to generate individual BAM files corresponding to different barcode combinations in the run manifest; (4) combining demultiplexed output by sample name and/or patient identifier; (5) removing barcodes from sequences and generate individual sample FASTQ files; (6) aligning sequences to barcodes; trimming the barcodes (e.g., using a PacBio trim script; (7) converting BAM files to FASTQ files (e.g., using bamtools); (8) copying FASTQ and CCS BAM files to final location; (9) and copying FASTQ files and the corresponding run manifest to a drop location to trigger CLC Workflow.

The CLC Analysis workflow was performed using the following steps: (1) An NGS data analysis workflow is executed on each sample using a current validated CLC Genomics Server version; (2) For each sample's FASTQ file: (a) reads were filtered to retain reads of 250-5000 bp length; (b) reads were aligned to the SARS-CoV-2 reference genome (“NC_045512v2”) using minimap2 to generate a BAM file; (c) local realignment was performed and variant calls made using the Low Frequency Variant Detection tool in CLC Genomics Server; and (d) both the assembly (BAM file) and detected variants (cf) were input into a downstream post-processing analysis. A script detected CLC process completion, initiating the launch of downstream analysis for samples in each SMRTcell.

Downstream Analysis

An example flow-chart for downstream (post-processing) analysis is shown in FIG. 5. Post-processing part 1 is represented in the first block in FIG. 5. The steps for post-processing part 1 were as follows. (1) Using the appropriate reference file VCFCons was used to generate the consensus sequences based on sequence alignment and variant calls for each sample. For this analysis, a minimum coverage of 4 CCS reads and minimum alternate frequency of 0.5 was required to assign a base to each genomic position and positions that did not satisfy this criterion were assigned an ambiguous base “N.” (2) Seqtk was used to generate the sequence base compositions, which was used later to determine the percentage of non-ambiguous bases. (3) Nextclade was used to generate the following using the consensus sequence as the input: (a) clade assignments; (b) mutation calling and (c) sample sequence quality check. (4) Pangolin was then used to assign lineages to the consensus sequence by generating the SARS-CoV-2 lineages, (known as the Pango nomenclature), then assigning a SARS-CoV-2 genome sequence lineage (Pango lineage). Pangolin only considers genomes that have at least 50% non-ambiguous bases. (5) SummaryStat was used to compile results from Nextclade, Pangolin, and Seqtk and generate coverage statistics needed for later QC, including mean of median amplicon coverage and percent genome coverage. For this analysis, the median coverage of the bases in 29 overlapping 1.2kb regions that span the entire SARS-CoV-2 genome were calculated for each of the samples. Statistics of the distribution of these coverage values (minimum, 1st quantile, mean, median, 3rd quantile and maximum) were calculated for each sample. Also, the percent genome coverage was calculated as the number of non-ambiguous bases (A, T, C, G) divided by the total sequence length, and lineage classifications are aggregated and only samples that produced a Nextclade result and Pangolin lineage call were retained for further processing.

At this point post-processing part 2 was initiated as shown for the “Combine Patient Metadata”, “Quality checking”, and “Generate final report” blocks in FIG. 5. Thus, again using the appropriate reference file strain surveillance-specific metadata (demographic data, percent genome coverage, and Ct values from the RT-PCR assay) QC was performed and the data added to the results. Samples that were missing metadata were dropped from the result set. Also, non-template QC was performed based on the non-template control. If the mean of the median coverage of the 29 genomic regions was >10 CCS reads, then all samples sequenced on the same plate were removed. Finally, coverage QC was performed. Samples with genome coverage >=90% were retained in the results, and samples with mean of median coverage >10 CCS reads were retained in the results. The results were then transferred to a Report System location for generating patient reports with corresponding Pangolin lineages. Samples that failed to produce a result were reported as: no lineage was able to be determined. SARS-CoV-2 virus detected, no lineage information can be reported.

The lineage calling criteria were as follows. Inclusion criteria: (1) CT <31; (2) corresponding metadata (strain surveillance); (3) >90% genome coverage; (4) mean of median coverage >10 CCS reads; (4) passing NTC control; and (5) Nextclade result and Pangolin lineage call. Exclusion criteria: (1) CT >31; (2) missing metadata (strain surveillance); (3) <90% genome coverage; (4) mean of median coverage <10 CCS reads; and (4) failing NTC control.

### Example 5

### Assessment of Potential New Variants and Classification

Revalidating the classification accuracy of the Virseq assay in response to the emergence of new variants (i.e. lineages) of the SARS-CoV-2 virus and concomitant changes to the pangolin classification software was performed as outlined in FIG. 6. The pangolin software is distributed through Dockerhub (at hub.docker.com/r/staphb/pangolin). The Pangolin site was monitored and checked by downloading and installing an updated docker container at regular intervals (e.g., weekly) for updates. If there were no updates, it was deemed that no action was required. The document docker container was updated in a change log along with the release notes. The updated docker files contained change notes and the latest versions of pangolin, pangoLEARN, pango-designation, scorpio, and constellations (see, github.com/cov-lineages).

If there were updates, a regression analysis was performed using in-house laboratory data. Essentially the steps were performed as follows. The new pangolin version was used to determine the lineage of samples contained within the reference set of historical Virseq sequences. The reference set included an initial SMRT cell from October 2021, predominantly composed of Delta lineages. It also contained two updates of Omicron lineages made in December 2021 and March 2022. Each sample in the reference set included its consensus sequence as well as the history of its lineage classifications made by previous pangolin versions. The reference set was updated periodically to include samples representing newer, more prevalent lineages as pangolin versions are updated.

Next, the format of the pangolin software output was compared with that of the previous version to determine if there are changes in the pangolin output format. If there were any changes to the CSV output (i.e. additional columns, changes in column names), these were documented and the laboratory Virseq pipeline modified as needed to accommodate the change. The modified version was then deployed to the QA environment for testing.

Next, any changes in lineage calls were assessed and compared with those expected from the software update change notes. Expected changes typically include reassignment among sublineages. If there were any unexpected changes in lineages (e.g. Delta sublineage to Alpha), these were investigated in detail and documented.

The acceptance criteria and action taken were as follows. Lineage classification disagreements are mostly due to the improvement of pangoLEARN/pango-designation definitions of the variants in the newer version. Most of these are sublineage reassignments but could also be due to changes in the model's defining variants. The sublineage reassignments were reviewed to ensure they are the expected changes under a parent lineage such as reassignment among AY in the parent Delta lineage. Another source of discordance could occur in samples with <90% genomic coverage. Any discordances that could be explained by sublineage reassignments or genome coverage issues as described above were documented and further reviewed for approval. The GISAID regression test was then performed. When discordances could not be explained as above and no new pangolin lineages have been added in the upgrade, the upgrade was halted and production continued with the current version of pangolin. The discordances were documented and stored with the updates as described above. Discordances were further investigated as new information became available and documented, or initiation of this protocol for the next release of pangolin could resolve discordance.

At this point, a second regression test was performed using publicly available (GISAID) sequences and their metadata. The latest GISAID sequences were downloaded and the metadata and pangolin lineages for all GISAID sequences obtained and the list of VOCs and VOIs updated based on WHO updates and the latest complete list of lineages. Next, a data simulator was used to model the coverage and error properties of the Virseq assay. The simulator used GISAID sequences as starting points and imposed simulated coverage and errors based on empirical coverage profiles and max-minor-allele frequencies from a collection of Virseq samples. The resulting simulated samples were run through pangolin, and the lineage classifications were compared to those of the original GISAID sequences. Classification stability was defined as the rate at which mutated sequences maintain their expected lineage classifications. In this regression test, two experiments were run to assess classification stability via simulation. First, up to 100 GISAID sequences were randomly sampled for each VOC/VOI to assess the classification stability of these important lineages, regardless of their frequency in the sequencing data available. This allowed an assessment of classification stability of emerging variants as well as new sublineages of existing ones. Second, 10,000 GISAID sequences from the database were randomly sampled for a frequency-based retrospective analysis of lineage classification stability. This allowed stability to be quantified relative to historical prevalence.

At this point, the output of the data simulator experiments was reviewed, checking for unexpected changes in classification stabilities with respect to previous regression tests using GISAID data for all known VOC/VOIs and the retrospective GISAID data. Any unexpected instabilities were investigated and documented. The upgrade was then accepted upon satisfying certain parameters. In some cases, the upgrade was requested if the median VOC NOI concordance between the simulated data and reference sequence was at least 90%. In cases where these criteria were not met, additional investigation was indicated.

If the new discordant lineage(s) were novel, the novel lineage(s) were tested to determine if they were detected using the methods disclosed herein. If the discordant variant(s) were not novel variant(s), they were investigated to find the root cause of discordance. This involved looking at the coverage of the reference sequence as well as the simulated sequences to ensure there was not an undesirable drop in base coverage in specific regions. Also, the simulation was re-run with another seed to determine if the discordance was reproduced. If it was, the upgrade was halted.

At this point the novel variants were assessed using the methods disclosed herein. For successful surveillance of emerging variants (lineages), the potential impact on the molecular loop inversion probe amplification was reviewed by conducting an in silico analysis as for example by identifying the location of the individual sequence variants in the emerging lineages and the associated molecular loop probes to assess the potential for interference in probe binding. For example, a very conservative estimate that the novel sequence variant overlapping with any probe will impact hybridization would then be used. Also, all adjacent probes in the region were reviewed to ensure coverage of the novel sequence variant. For any sequence variant that could result in a reduction of coverage within a particular region, the impacted probes within the pangolin lineage update validation summary were documented.

### Example 6

### Embodiments

The disclosure may be better understood by reference to the following non-limiting embodiments.
A1. A method for identifying and/or tracking variants of SARS-CoV-2 comprising:

(a) identifying a sample from a subject as positive for SARS-CoV-2 nucleic acid and/or antibodies to SARS-CoV-2;

(b) generating a sample-specific SARS-CoV-2 nucleic acid from the sample;

(c) performing nucleic acid sequencing on the sample-specific SARS-CoV-2 nucleic acid; and

(d) determining whether the nucleic acid sequence comprises a SARS-CoV-2 variant sequence.
A2. A method of any one of the previous or subsequent method embodiments, wherein generating a sample-specific SARS-CoV-2 nucleic acid comprises using reverse transcriptase polymerase chain reaction (RT-PCR) to generate a sample-specific SARS-CoV-2 cDNA.
A3. A method of any one of the previous or subsequent method embodiments, wherein the SARS-CoV-2 cDNA is then further amplified using tiled primers that bind at spaced intervals along the viral genome.
A4. A method of any one of the previous or subsequent method embodiments, wherein the tiled primers are spaced such that adjacent primers are approximately 600 bp apart from each other.
A4.1 A method of any one of the previous or subsequent method embodiments, wherein further comprises hybridizing one strand of the sample SARS-CoV-2 cDNA to a single-stranded probe DNA template comprising a pair of SARS-CoV-2 probes, wherein the first probe is positioned at the 3′ end of the probe DNA template to function as a forward primer and the second probe is positioned at the 5′ end of the probe DNA template to function as a reverse primer.
A4.2 A method of any one of the previous or subsequent method embodiments, wherein the SARS-CoV-2 genome is amplified in a highly efficient manner regardless of the presence or absence of new variants.
A4.3 A method of any one of the previous or subsequent method embodiments, wherein the tiled primers are primers further comprise an adaptor for the addition of a barcode sequence used to correlate the SARS-CoV-2 sample-specific nucleic acid to a sample number and/or universal primer sites for nucleic acid sequencing.
A5. A method of any one of the previous or subsequent method embodiments, wherein the single-stranded probe DNA template further comprises universal sequencing primers positioned internal to the probe sequences.
A6. A method of any one of the previous or subsequent method embodiments, wherein the single-stranded probe DNA template further comprises an adaptor sequence for the addition of a barcode sequence used to correlate the SARS-CoV-2 sample-specific nucleic acid to a sample number.
A7. A method of any one of the previous or subsequent method embodiments, further comprising filling in the sequence between the two probes to generate a circular single-stranded probe DNA template comprising sequence specific to the sample SARS-CoV-2 cDNA between the two probe sequences.
A8. A method of any one of the previous or subsequent method embodiments, further comprising releasing the circular single-stranded probe DNA template comprising sequence specific to the sample SARS-CoV-2 cDNA from the sample-specific SARS-CoV-2 DNA.
A9. A method of any one of the previous or subsequent method embodiments, further comprising digestion of the circular single-stranded probe DNA template comprising sequence specific to the sample SARS-CoV-2 cDNA to generate a linear DNA used as a template for the step of performing nucleic acid sequencing on the sample-specific SARS-CoV-2 nucleic acid.
A10. A method of any one of the previous or subsequent method embodiments, further comprising uploading the results of the step of determining whether the nucleic acid sequence comprises a SARS-CoV-2 variant sequence into a depository for further classification if a variant is detected.
A11. A method of any one of the previous or subsequent method embodiments, wherein the depository is a CDC database.
A12. A method of any one of the previous or subsequent method embodiments, wherein the nucleic acid sequencing comprises sequencing at least 80%, or optionally 85%, or optionally 90% of the entire viral genome.
A13. A method of any one of the previous or subsequent method embodiments, further comprising identifying the geographic location of the subject.
A14. A method of any one of the previous or subsequent method embodiments, wherein the nucleic acid sequencing comprises whole genome sequencing.
A15. A method of any one of the previous or subsequent method embodiments, wherein the determining whether the nucleic acid sequence comprises a SARS-CoV-2 variant sequence comprises aligning the sample SAR-CoV-2 sequence to a SARS-CoV-2 reference genome to generate a sample-specific assembly and consensus sequence.
A15.1 A method of any one of the previous or subsequent method embodiments, wherein a sample SAR-CoV-2 nucleic acid sequence having a minimum coverage of at least 50% is used as the input for calling variants and/or for generating a sample-specific genome assembly to generate a consensus sequence for each sample.
A15.2 A method of any one of the previous or subsequent method embodiments, wherein there is a defined threshold for generating the consensus sequence.
A15.3 A method of any one of the previous or subsequent method embodiments, wherein the defined threshold includes at least 4 circular consensus sequencing (CCS) reads covering an individual base pair and/or an alternate allele frequency compared to the reference of >50%.
A15.4 A method of any one of the previous or subsequent method embodiments further comprising evaluation of an external no template control (NTC) and/or an external positive template control (PTC) to assess the validity of the results
A15.5 A method of any one of the previous or subsequent method embodiments, wherein the sample SAR-CoV-2 nucleic acid sequencing reads are filtered to retain reads of 250-5000 bp length.
A15.6 A method of any one of the previous or subsequent method embodiments, wherein the sample SAR-CoV-2 nucleic acid sequencing reads are aligned to the SARS-CoV-2 reference genome (NC_045512v2).
A15.7 A method of any one of the previous or subsequent method embodiments, wherein after the sample SAR-CoV-2 nucleic acid sequencing reads are aligned to the SARS-CoV-2 reference genome local realignment is performed and variant calls made.
A15.8 A method of any one of the previous or subsequent method embodiments, wherein a determination of the sample SAR-CoV-2 nucleic acid sequence base composition is generated to determine the percentage of non-ambiguous bases.
A15.9 A method of any one of the previous or subsequent method embodiments, wherein any one or all of the following may optionally be generated using the consensus sequences as the input: (a) a clade assignment; (b) a determination of a mutation and (c) a sample sequence quality check.
A16. A method of any one of the previous or subsequent method embodiments, wherein step (d) of determining whether the nucleic acid sequence comprises a SARS-CoV-2 variant sequence further comprises assessing the lineage for the sample.
A.16.1 A method of any one of the previous or subsequent method embodiments, wherein lineages are assigned to the consensus sequence by generating the SARS-CoV-2 lineages, then assigning a SARS-CoV-2 genome sequence lineage.
A16.2 A method of any one of the previous or subsequent method embodiments, wherein lineage assignment is set so as only to consider genomes that have at least 50% non-ambiguous bases.
A16.3 A method of any one of the previous or subsequent method embodiments, wherein strain lineage results are released for samples with 90% genome coverage.
A16.4 A method of any one of the previous or subsequent method embodiments, wherein strain lineage results are released for samples having a mean of median read coverage across the whole genome is >10 circular consensus sequence (CCS) reads.
A16.5 A method of any one of the previous or subsequent method embodiments, wherein the different CCS read metrics are based on the nucleotide level (4 CCS reads) and on the genome level (10 CCS reads).
A16.6 A method of any one of the previous or subsequent method embodiments, wherein Pangolin is used to assign lineages.
A16.7 A method of any one of the previous or subsequent method embodiments, further comprising generating coverage statistics.
A16.8 A method of any one of the previous or subsequent method embodiments, wherein the coverage statistics are generated using SummaryStat.
A16.9 A method of any one of the previous or subsequent method embodiments, wherein the median coverage of the bases in 29 overlapping 1.2 kb regions that span the entire SARS-CoV-2 genome are calculated for each of the samples
A16.10 A method of any one of the previous or subsequent method embodiments, wherein the mean of the median coverage of the 29 genomic regions is >10 CCS reads.
A16.11 A method of any one of the previous or subsequent method embodiments, wherein samples with genome coverage >=90% are retained in the results.
A16.12 A method of any one of the previous or subsequent method embodiments, wherein samples with mean of median coverage >10 CCS reads are retained in the results.
A16.13 A method of any one of the previous or subsequent method embodiments, wherein using demographic data, percent genome coverage, and Ct values from the RT-PCR assay, QC is performed and the data added to the results.
A16.14 A method of any one of the previous or subsequent method embodiments, wherein the results are used to generate patient reports with corresponding lineages and/or geographic assignments.
A16.15 A method of any one of the previous or subsequent method embodiments, wherein inclusion criteria include: (1) CT <31; (2) corresponding metadata (strain surveillance); (3) >90% genome coverage; (4) mean of median coverage >10 CCS reads; (4) passing NTC control; and (5) and lineage call.
A16.16 A method of any one of the previous or subsequent method embodiments, wherein exclusion criteria include: (1) CT >31; (2) missing metadata (strain surveillance); (3) <90% genome coverage; (4) mean of median coverage <10 CCS reads; and (4) failing NTC control.
A17. A method of any one of the previous or subsequent method embodiments, further comprising revalidating the lineage assignments by determining if an update to the depository has been made
A17.1 A method of any one of the previous or subsequent method embodiments, wherein revalidating is performed prior to the step of determining whether the nucleic acid sequence comprises a SARS-CoV-2 variant sequence.
A17.2 A method of any one of the previous or subsequent method embodiments, wherein the revalidation includes a regression analysis using in-house data to determine if a previously assigned lineage should be changed.
A17.3 A method of any one of the previous or subsequent method embodiments, wherein the in-house data comprises data sets defined by at least one of date of accrual, SARS-CoV-2 lineage, geographic origin of the sample, history of lineage classification, or updates to algorithm used for lineage classification.
A17.4 A method of any one of the previous or subsequent method embodiments, wherein the update includes a change to a lineage or sublineage for in-house data.
A17.5 A method of any one of the previous or subsequent method embodiments wherein the revalidation includes a regression analysis using data from a depository.
A17.6. A method of any one of the previous or subsequent method embodiments wherein the depository is GISAID.
A17.7 A method of any one of the previous or subsequent method embodiments, wherein GISAID sequences are downloaded and the metadata and lineages for all GISAID sequences obtained and the list of VOCs and VOIs updated based on WHO updates and the latest complete list of lineages.
A17.8 A method of any one of the previous or subsequent method embodiments further comprising using a data simulator to model the coverage and error properties of the in-house assay.
A17.9 A method of any one of the previous or subsequent method embodiments, wherein the simulator uses GISAID sequences as starting points and imposes simulated coverage and errors based on empirical coverage profiles and max-minor-allele frequencies from a collection of samples, the resulting simulated samples are run through the lineage algorithm, and the lineage classifications are compared to those of the original GISAID sequences.
A17.10 A method of any one of the previous or subsequent method embodiments, wherein classification stability is defined as the rate at which mutated sequences maintain their expected lineage classifications.
A17.11 A method of any one of the previous or subsequent method embodiments, wherein for the simulation 100 GISAID sequences are randomly sampled for each VOC and/or VOI.
A17.12 A method of any one of the previous or subsequent method embodiments, wherein for the simulation 10,000 GISAID sequences from the database are randomly sampled for a frequency-based retrospective analysis of lineage classification stability.
A17.13 A method of any one of the previous or subsequent method embodiments, wherein the upgrade is requested if the median VOC/VOI concordance between the simulated data and reference sequence is at least 90%
A18. A method of any one of the previous or subsequent method embodiments, wherein at least some of the steps are controlled by a computer and/or a computer-program product tangibly embodied in a non-transitory machine-readable storage medium,
A18.1 A method of any one of the previous or subsequent method embodiments, wherein at least some of the steps are controlled by:

one or more data processors; and

a non-transitory computer readable storage medium containing instructions which, when executed on the one or more data processors, cause the one or more data processors to perform processing comprising any of the method steps.
B1. A system comprising at least one station or component for performing any of the previous or subsequent method embodiments.
B2. A system comprising at least one station or component for performing any of the previous or subsequent method embodiments comprising:

(a) identifying a sample from a subject as positive for SARS-CoV-2 nucleic acid and/or antibodies to SARS-CoV-2;

(b) generating a sample-specific SARS-CoV-2 nucleic acid from the sample;

(c) performing nucleic acid sequencing on the sample-specific SARS-CoV-2 nucleic acid; and

(d) determining whether the nucleic acid sequence comprises a SARS-CoV-2 variant sequence.
B3. A system of any one of the previous or subsequent embodiments, wherein at least some of the steps are controlled by a computer and/or a computer-program product tangibly embodied in a non-transitory machine-readable storage medium.
B.4 A system of any one of the previous or subsequent method embodiments, wherein at least some of the steps are controlled by:

one or more data processors; and

a non-transitory computer readable storage medium containing instructions which, when executed on the one or more data processors, cause the one or more data processors to perform processing comprising any of the method steps.
C1. A computer-program product tangibly embodied in a non-transitory machine-readable storage medium, when executed on the one or more data processors, cause the one or more data processors to perform processing comprising:

(a) identifying a sample from a subject as positive for SARS-CoV-2 nucleic acid and/or antibodies to SARS-CoV-2;

(b) generating a sample-specific SARS-CoV-2 nucleic acid from the sample;

(c) performing nucleic acid sequencing on the sample-specific SARS-CoV-2 nucleic acid; and

(d) determining whether the nucleic acid sequence comprises a SARS-CoV-2 variant sequence.
D1. A computer-program product tangibly embodied in a non-transitory machine-readable storage medium, including instructions configured to run at least one station or component of a system for performing any of the steps of:

(a) identifying a sample from a subject as positive for SARS-CoV-2 nucleic acid and/or antibodies to SARS-CoV-2;

(b) generating a sample-specific SARS-CoV-2 nucleic acid from the sample;

(c) performing nucleic acid sequencing on the sample-specific SARS-CoV-2 nucleic acid; and

(d) determining whether the nucleic acid sequence comprises a SARS-CoV-2 variant sequence.

That which is claimed:

1. A method for identifying and/or tracking variants of SARS-CoV-2 comprising:
(a) identifying a sample from a subject as positive for SARS-CoV-2 nucleic acid and/or antibodies to SARS-CoV-2;
(b) generating a sample-specific SARS-CoV-2 cDNA from the sample;
(c) performing nucleic acid sequencing on the sample-specific SARS-CoV-2 nucleic acid; and
(d) determining whether the nucleic acid sequence comprises a SARS-CoV-2 variant sequence.

2. The method of claim 2, wherein the SARS-CoV-2 cDNA is then further amplified using tiled primers that bind at spaced intervals along the viral genome.

3. The method of claim 2, wherein the tiled primers are spaced such that adjacent primers are approximately 600 bp apart from each other.

4. The method of claim 2, wherein generating a sample-specific SARS-CoV-2 nucleic acid further comprises hybridizing one strand of the sample SARS-CoV-2 cDNA to a single-stranded probe DNA template comprising a pair of SARS-CoV-2 probes, wherein the first probe is positioned at the 3′ end of the probe DNA template to function as a forward primer and the second probe is positioned at the 5′ end of the probe DNA template to function as a reverse primer.

5. The method of claim 4, wherein the single-stranded probe DNA template further comprises universal sequencing primers positioned adjacent to the probe sequences.

6. The method of claim 4, wherein the single-stranded probe DNA template further comprises an adaptor sequence for the addition of a barcode sequence used to correlate the SARS-CoV-2 sample-specific nucleic acid to a sample number.

7. The method of claim 6, wherein the barcode is linked to a zip code or other geographic identifier for the sample.

8. The method of claim 4, further comprising filling in the sequence between the two probes to generate a circular single-stranded probe DNA template comprising sequence specific to the sample SARS-CoV-2 cDNA between the two probe sequences.

9. The method of claim 1, wherein the nucleic acid sequencing comprises sequencing at least 90% of the entire viral genome.

10. The method of claim 1, wherein the median coverage of bases in 29 overlapping 1.2 kb regions that span the entire SARS-CoV-2 genome are calculated for each of the samples.

11. The method of claim 1, further comprising uploading the results of step (d) into a depository for further classification if a variant is detected.

12. The method of claim 11, wherein the depository is a CDC database.

13. The method of claim 1, further comprising identifying the geographic location of the subject.

14. The method of claim 1, wherein the determining whether the nucleic acid sequence comprises a SARS-CoV-2 variant sequence comprises aligning the sample SAR-CoV-2 sequence to a SARS-CoV-2 reference genome to generate a sample-specific assembly and consensus sequence.

15. The method of claim 1, wherein step (d) further comprises assessing the lineage for the sample.

16. The method of claim 1, wherein inclusion criteria for step (d) include: >90% genome coverage, a mean of median coverage >10 CCS reads; and lineage determination for the sample.

17. The method of claim 11, further comprising determining if an update to the depository has been made prior to the step of determining whether the nucleic acid sequence comprises a SARS-CoV-2 variant sequence.

18. The method of claim 1, wherein at least some of the steps are controlled by:
one or more data processors; and
a non-transitory computer readable storage medium containing instructions which, when executed on the one or more data processors, cause the one or more data processors to perform processing comprising any of the method steps.

19. A system comprising:
one or more data processors; and
a non-transitory computer readable storage medium containing instructions which, when executed on the one or more data processors, cause the one or more data processors to perform processing comprising:
(a) identifying a sample from a subject as positive for SARS-CoV-2 nucleic acid and/or antibodies to SARS-CoV-2;
(b) generating a sample-specific SARS-CoV-2 nucleic acid from the sample;
(c) performing nucleic acid sequencing on the sample-specific SARS-CoV-2 nucleic acid; and
(d) determining whether the nucleic acid sequence comprises a SARS-CoV-2 variant sequence.

20. A computer-program product tangibly embodied in a non-transitory machine-readable storage medium, including instructions configured to cause one or more data processors to perform processing comprising:
(a) identifying a sample from a subject as positive for SARS-CoV-2 nucleic acid and/or antibodies to SARS-CoV-2;
(b) generating a sample-specific SARS-CoV-2 nucleic acid from the sample;
(c) performing nucleic acid sequencing on the sample-specific SARS-CoV-2 nucleic acid; and
(d) determining whether the nucleic acid sequence comprises a SARS-CoV-2 variant sequence.
